(19) United States
(12) Patent Application Publication
(10) Pub. No.: US 2017/0360073 A1
(43) Pub. Date: Dec. 21, 2017

(54) OPTIMIZED NUTRIENT FATTY ACID COMPOSITION

(71) Applicant: JOLLY MATHEW VADAKKEMURI, ERNAKULAM (IN)

(72) Inventor: JOLLY MATHEW VADAKKEMURI, ERNAKULAM (IN)

(21) Appl. No.: 15/731,247

(22) PCT Filed: Nov. 9, 2015
(86) PCT No.: PCT/IN2015/000412

§ 371 Date: May 9, 2017

(30) Foreign Application Priority Data
Nov. 7, 2014 (IN) 5623/CHE/2014

(51) Int. Cl. A23L 33/12; A23D 9/00; A61Q 17/04; A61Q 5/00; A61Q 19/02; A23K 20/158; A61K 8/36; A61Q 19/06; A23L 33/15; A61Q 7/00

(52) U.S. Cl. CPC A23L 33/12; A23L 33/15; A23D 9/00; A61Q 17/04; A61Q 7/00; A61Q 19/02; A23K 20/158; A61K 8/361; A61Q 19/06; A61Q 5/00; A23V 2002/00; A23V 2250/1882; A61K 2800/92

(57) ABSTRACT

An optimized nutrient fatty acid composition capable of using as food, skin care, hair care and media for delivery of drugs and nutrients through mucous membrane, skin and parenterally for humans, pets and farm animals, with a ratio of omega 6 fatty acid to omega 3 fatty acids ranging from 1:1 to 1:1.25 stabilized with vitamin E 0.8-4 mg/ml. More specifically a stable fatty acid composition with 50-70% of the total fatty acids as essential and, or semi essential fatty acid of which 25-35% omega 3 fatty acids, 25-35% as omega 6 fatty acids and 20-30% as monounsaturated fatty acids is capable of optimization of bodily functions, maintenance of health, prevention of diseases, promotion of healing, delay aging, and treatment of diseases caused by imbalance in omega 3 and 6 fatty acid ratio in diet and nutritional support for pregnant and lactating women, growing children, senior citizens, sports persons, obese and over weight on calorie restricted diet.

### FIELD OF THE INVENTION

This invention relates to an optimized nutrient fatty acid composition capable of using as food, skin care, hair care and media for delivery of drugs and nutrients through mucous membrane, skin and parenterally for humans, pets and farm animals, with a ratio of omega 6 fatty acid to omega 3 fatty acids ranging from 1:1 to 1:1.25. Another embodiment of the invention protects the unstable poly unsaturated essential and semi essential fatty acids of omega 6 and 3 fatty acids. Method of stabilization of unsaturated fatty acid by adding vitamin E in the range of 0.8-4 mg/ml of the composition to protect the unsaturated fatty acids from lipid peroxidation in vitro and in vivo and to avoid rancidity. The amount of vitamin E needed is determined based on the formula (% R1×1+% R2×2+% R3×3+% R4×4+% R5×5+% R6×6) divided by the factor F for calculation.

This optimized nutrient fatty acid composition for use as a food, a food ingredient, a food supplement, a cooking oil, a skin care oil, a hair care oil, a carrier oil for nutrients and drugs, a base oil for ointments, creams and lotions for cosmetic and pharmaceutical preparations and a base oil for preparation of intravenous lipid emulsion. The preferred embodiment of the invention is a stable fatty acid composition with 50-70% of the total fatty acids as essential and or semi essential fatty acids of which 25-35% omega 3 fatty acid, 25-35% as omega 6 fatty acid and 20-30% as monounsaturated fat is capable of optimization of bodily functions, maintenance of health, prevention of diseases, promotion of healing, delay aging, and treatment of diseases caused by imbalance in omega 3 and 6 fatty acid ratio in diet including heart disease, hypertension, type 2 diabetes, cancer, mental disorders, immunity related diseases, inflammatory diseases, asthma, allergy, eczema, arthritis psoriasis and nutritional support for pregnant and lactating women, growing children, sports persons, obese and over weight on calorie restricted diet.

### Prior Art and Problem to be Solved

During the advancement of civilization and industrialization human diet and nutrition has changed drastically over the last century. Increased production and consumption of oil seeds and oil increased the dietary intake of linoleic acid an essential fatty acid of omega 6 fatty acid group. This lead a change in omega 6 and 3 fatty acid ratio in the diet of human from around 1:1 to 10-40:1. This change has a major role in the pathogenesis of the so called diseases of civilization/life style diseases including heart disease, hypertension, type 2 diabetes and inflammatory diseases.

Advancement in medical science developed many drugs and surgical procedures to address the life style diseases. A major number of drugs used in the modern medical field are giving its benefits and also its side effects by acting in the metabolic pathway of essential fatty acids omega 6 LA and omega 3 ALA derived ecosanoids and decosanoids. These omega 3 and omega 6 group of ecosanoids and decosanoids acts through more than twenty signaling pathways which control bodily functions involving inflammation, cell growth and central nervous system. These two groups of molecules has complex mechanism of action, continuously competing each other for the production and action. So a drug targeted to reduce inflammation on lungs for asthma may alter the balance in the central nervous system and produce serious side effects to the medication or a drug targeted to act on joints for arthritis may produce serious side effects on cardiovascular system. Many medicines used in modern medicine acts through these pathways which block or enhance the action of both these functionally opposite omega 3 and omega 6 pathways which is not desirable and cause side effects related to the medication. Many drugs of this group were withdrawn from market due to serious side effects after few years in the market. Generally omega 3 fatty acid and its metabolites are anti-inflammatory in nature and omega 6 fatty acid and its metabolites are pro inflammatory in nature. Functions of these omega 3 and 6 signaling molecules varies according to the type of cells and organ systems which further complicates the problem of imbalance in this two essential fatty acids and the drugs targeted to act in the pathway of their metabolites.

The feeder food of farm animals with high omega 6 content has changed the ratio of omega 6 and 3 fatty acids in milk, meat and egg. Increase in pro inflammatory omega 6 fatty acid consumption and no corresponding increase, or a decrease in omega 3. fatty acid consumption and increase in pollution which act as a trigger for inflammatory and immunoreactions lay base to the pathogenesis of modern life style diseases. Maintaining a balance in consumption of these two essential omega 6 and omega 3 fatty acids in the diet is essential for growth, development, maintenance of health, recovery from diseases, and healing of wounds.

Cleansing products like soaps, shampoo and detergents wash away the protective sebaceous secretions of the skin and exposes the skin to environmental pollutants. Damage in the ozone layer of the atmosphere brings more harmful rays to earth with its negative effects on the skin.

Currently available cosmetic and pharmaceutical topical preparations, oils, ointments, creams and lotions are commonly based on petroleum byproducts which have no nutritive value and their absorption into the body is not desirable. Hence there is need for correction of the essential fatty acid ratio in the diet, a body and hair care preparation which nourishes the skin cells, hair follicles and protects the skin form harmful radiation of sun and pollution. It is also ideal to have a safe and effective media for better absorption of medicines and nutrients applied topically.

### OBJECTS OF THE INVENTION

It has already been proposed the need for correction of the essential fatty acid ratio in the diet, a body and hair care preparation which nourishes the skin cells, hair follicles and protects the skin form harmful radiation of sun and pollution. It is ideal to have a safe and effective media for better absorption of medicines and nutrients applied topically.

The main objects of this invention is to provide an optimized nutrient fatty acid composition which can provide simple, safe, practical and economic solution for correcting the dietary imbalance in the essential fatty acid intake of humans, pets and farm animals, thereby bring reduction in related diseases and reduce the intake of drugs and associated side effects.

Another object of this invention is to provide skin and hair care composition which will provide nutrition to skin and hair follicle and protect the skin from pollutants, infections and harmful rays.

Yet another object of this invention is to provide a safe media for delivery of drugs and nutrients through skin and mucous membrane which promotes absorption of nutrients and drugs so that it can be used as a base for oil, ointment, cream and lotion which will deliver nutrients and medicines locally thereby reduce the dose of medicine and systemic side effects.

### SUMMARY OF THE INVENTION

The preferred embodiment of invention is an optimized nutrient fatty acid composition capable of using as food, skin care, hair care and media for delivery of drugs and nutrients through mucus membrane, skin and parenterally for humans, pets and farm animals, with a ratio of omega 6 fatty acid to omega 3 fatty acids ranging from 1:1 to 1:1.25. Another embodiment of the invention is considering the unstable nature of poly unsaturated essential and semi essential fatty acids of omega 6 and 3 fatty acids, a method of stabilization of unsaturated fatty acid by adding vitamin E in the range of 0.8-4 mg/ml of the composition to protect the unsaturated fatty acids from lipid peroxidation in vitro and in vivo and to avoid rancidity. The amount of vitamin E needed is determined based on the formula (% R1×1+% R2×2+% R3×3+% R4×4+% R5×5+% sR6×6) divided by the factor F for calculation, wherein % R1 is the percentage of fatty acid with one double bond in the fatty acid chain, % R2 is the percentage of fatty acid with two double bonds in the fatty acid chain, % R3 is the percentage of fatty acid with three double bonds in the fatty acid chain, % R4 is the percentage of fatty acid with four double bonds in the fatty acid chain, % R5 is the percentage of fatty acid with five double bonds in the fatty acid chain, % R6 is the percentage of fatty acid with six double bonds in the fatty acid chain, the factor F is an arbitrary number 150.

The optimized fatty acid composition containing 50-70% of the total fatty acids as essential or semi essential fatty acids wherein 25-35% as omega 3 fatty acids, 25-35% as omega 6 fatty acids and stabilized with vitamin E in the range of 0.8-4 mg/ml.

The type of fatty acids in the composition is selected considering the properties needed for the end use from a group including ALA (alpha linoleic acid), SDA (steriodonic acid), ETA (ecosatetraenoic acid), EPA (ecosapentanoic acid), DPA (decosapentaenoic acid), DHA Decosahexanoic acid), LA (linoleic acid), GLA (gamalinoleic acid), DGLA (dihomogamalinoleic acid) and AA (arachidonic acid), oleic acid, palmitoic acid, vaccenic acid, erucic acid and saturated fatty acids.

The source of fatty acids for the production depends on the oil content and the fatty acid profile of the source and the seasonal and geographical availability of fatty acid source. Any suitable source of fatty acids including plant source, genetically modified plant source, animal source, genetically modified animal source, marine life source, genetically modified marine life source, an algal source, a genetically modified algal source, a fungal source, a genetically modified fungal source, a microorganism and a genetically modified microorganism may be used for production. Production steps include cleaning and drying the selected ingredients, dehydrating the ingredients at 40-80 degree centigrade to avoid hydrolytic degradation of unsaturated fatty acids, extracting the oil from the selected raw materials using known technologies for the selected source materials, analyzing the oil samples for fatty acid composition, measuring and combining different oils considering the fatty acid analysis data to get the fatty acid composition with omega 6 fatty acid to omega 3 fatty acid ratio ranging from 1:1 to 1:1.25. Stabilization of the unsaturated fatty acids by adding vitamin E in the range of 0.8-4 mg/ml of the composition to protect the unsaturated fatty acids from lipid peroxidation in vitro and in vivo and to avoid rancidity. Then mixing the fatty acid composition in a stirrer at 200-500 RPM for 2-5 minutes to get an even mixture and packing the product in to air tight/nitrogen filled and opaque containers to avoid contact with oxygen and exposure to light.

This optimized nutrient fatty acid composition is ideal to use as a food, a food ingredient, a food supplement, a cooking oil, a skin care oil, a hair care oil, a carrier oil for nutrients and drugs, a base oil for ointments, creams and lotions for cosmetic and pharmaceutical preparations and a base oil for preparation of intravenous lipid emulsion. The preferred embodiment of the invention a stable fatty acid composition with 50-70% of the total fatty acids as essential and, or semi essential fatty acid of which 25-35% omega 3 fatty acid, 25-35% as omega 6 fatty acid and 20-30% as monounsaturated fats capable of optimization of bodily functions, maintenance of health, prevention of diseases, promotion of healing, delay aging, and treatment of diseases caused by imbalance in omega 3 and 6 fatty acid ratio in diet including heart disease, hypertension, type 2 diabetes, cancer, mental disorders, immunity related diseases, inflammatory diseases, asthma, allergy, eczema, arthritis, psoriasis and nutritional support for pregnant and lactating women, growing children, sports persons, obese and over weight on calorie restricted diet.

### BRIEF DESCRIPTION OF THE DRAWING

An exemplary embodiment of the present invention is illustrated by way of example in the accompanying drawings and the invention will become better understood when the following detailed description is read with reference to the accompanying drawings.

FIG. 1 shows a flow diagram depicting the essential fatty acid metabolism and interactions along with the site of action of a large number of drugs currently used as modern medicines coming under Cyclooxygenase (COX) and lipoxygenase (LOX) inhibitors, receptor antagonist and receptor modifying drugs used in the treatment of diseases including heart disease, hypertension, stroke, inflammation, allergy, asthma, arthritis, acidity, glaucoma, mental disorders, cancer and symptomatic relief of pain and fever there by explain the relevance and the theory behind this invention.

FIG. 2 shows a flow diagram depicting the various stages in the method of production of the optimized nutrient oil having optimized nutrient fatty acid composition according to an exemplary embodiment under the invention.

FIG. 3 shows a flow diagram depicting the production of optimized nutrient fat from the optimized nutrient oil having the optimized nutrient fatty acid composition according to an exemplary embodiment under the invention.

FIG. 4 shows a flow diagram depicting the production of skin care and hair care products from optimized nutrient oil having the optimized nutrient fatty acid composition according to an exemplary embodiment under the invention.

FIG. 5 shows a flow diagram depicting the production of pharmaceutical products from the optimized nutrient oil having the optimized nutrient fatty acid composition according to an exemplary embodiment under the invention.

FIG. 6 shows a flow diagram depicting the production of intravenous emulsion from the optimized nutrient oil having the optimized nutrient fatty acid composition according to an exemplary embodiment under the invention.

### DETAILED DESCRIPTION OF THE INVENTION

The preferred embodiment of invention is an optimized nutrient fatty acid composition as food, skin care, hair care and media for delivery of drugs and nutrients through mucus membrane, skin and parenterally for humans, pets and farm animals, with a ratio of essential and semi essential fatty acids of omega 6 fatty acid to omega 3 fatty acids ranging from 1:1 to 1:1.25. Further considering the unstable nature of poly unsaturated fatty acids, the composition is stabilized by adding vitamin E in the range of 0.8-4 mg/ml of the composition to protect the unsaturated fatty acids from lipid peroxidation in vitro and in vivo and to avoid rancidity.

Omega 6 and 3 fatty acid ratio in the diet of human has changed from around 1:1 to 10-40:1 with progress in civilization and industrialization. The anti-inflammatory omega 3 fatty acid in the diet is reducing and pro inflammatory omega 6 fatty acids level is increasing with advancement of industrialization. This change has a major role in the pathogenesis of life style diseases including heart disease, hypertension, behavioral abnormalities, type 2 diabetes, immunity related diseases and inflammatory diseases. With advancement of medical science many drugs and surgical procedures are developed to address these problems. A major number of drugs using in the modern medical field are giving its benefits and also it's side effects by acting in the metabolic pathway of essential fatty acids LA (linoleic acid) and ALA (alfa linoleic acid).

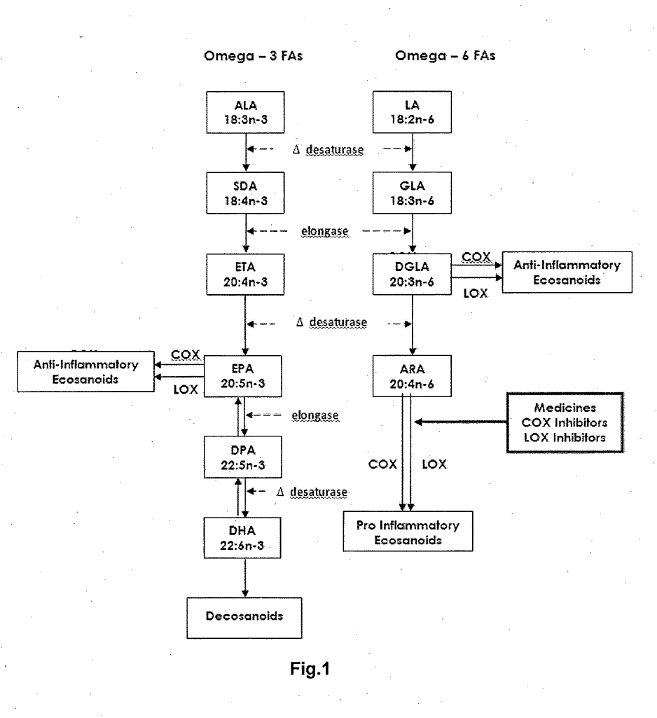

FIG. 1 explains the essential fatty acid pathway and its interactions and site of action of drugs and the mechanism by which the body is benefited at a cellular level by the optimized nutrient fatty acid composition in health and diseases. This explains essential fatty acids linoleic acid (LA) and alfa linolenic acid (ALA) metabolism and interaction of their metabolites along with the site of action of a large number of drugs currently used in modern medicine including cyclooxygenase (COX) and lipoxygenase (LOX) inhibitors; prostaglandins, prostacyclins and leucotrins receptor agonist, antagonist and receptor modifying drugs which act in the pathway of essential fatty acids LA& ALA and their metabolites for the treatment of diseases including heart disease, hypertension, stroke, inflammation, allergy, asthma, arthritis, acidity, glaucoma, cancer and symptomatic relief of pain and fever.

LOX and COX enzymes and their end product receptors are common for pro inflammatory ecosanoids from arachidonic acid and anti-inflammatory ecosanoids and decosanoids from GLA, EPA and DHA. Any drug used to block the ecosanoids from pro inflammatory arachidonic acid will block the anti-inflammatory ecosanoids production from GLA, EPA and DHA which is not desirable. These omega 3 and omega 6 group of ecosanoids and decosanoids acts through more than twenty signaling pathways which control bodily functions involving inflammation, cell growth and central nervous system. These two groups of molecules have complex mechanism of action, continuously competing with each other for production and action. So a drug targeted to reduce inflammation on lungs for asthma may alter the balance in the central nervous system and produce serious side effects to the medication or a drug targeted to act on joints for arthritis may produce serious side effects on cardiovascular system. Many medicines used in modern medicine act through these pathways which block or enhance the action of both these functionally opposite omega 3 and omega 6 pathways which is not desirable and cause side effects related to the medication. Many drugs of this group were withdrawn from market due to serious side effect after few years in the market. Generally omega 3 fatty acid and its metabolites are anti-inflammatory in nature and omega 6 fatty acid and its metabolites are pro inflammatory in nature. Functions of these omega 3 and 6 signaling molecules varies according to the type of cells and organ systems which further complicates the problem of imbalance in these two essential fatty acids and the drugs targeted to act in the pathway of their metabolites.

In the last century because of industrialization human, pet and farm animal food habit has changed especially in terms of fat consumption and the fatty acid composition in the consumed food. Dietary fat consist of heterogeneous mixture of triglycerides and small proportion of phospholipids, glycolipids, monoacylglycerol, diacylglycerols and unsaponifiable fraction composed of fat soluble chemicals collectively designated as non-glyceride components. Fatty acids are classified into 4 groups. 1) Saturated fatty acids (SFAs), 2) Monounsaturated fatty acids (MUFAs), 3) Omega 6 polyunsaturated fatty acids (omega 6 PUFAs), 4) Omega 3 polyunsaturated fatty acids (omega 3 PUFAs). Unsaturated fatty acids, MUFAs and PUFAs containing one or more double bonds in trans configuration are called trans fatty acids (TFAs). Partially hydrogenated fat manufactured for human consumption like dalda/vanaspathi contain high proportion of trans fat and studies show that it leads to health problems like cardio vascular diseases. Dietary fats have 4 major functions in the body. 1. Provide cushioning for the body and organs. 2. Energy for cellular activity and as energy store. 3. Building blocks of cell membrane. 4. Modulations of membrane structure and function. 5. Raw materials for production of cellular mediators-ecosanoids and decosanoids

Humans can synthesize SFAs and MUFAs besides obtaining from diet, while they cannot synthesize the parent omega 6 PUFA, namely linoleic acid (LA, 18:2 n-6) and parent omega 3 PUFA, Alpha linolenic acid (ALA, 18:3n-3). These two fatty acids have to be obtained from food and are called essential fatty acids.

A series of longer chain PUFAs (LCPUFAs) are produced from essential fatty acids LA and ALA by the action of consecutive chain elongase and desaturase enzymes. Arachidonic acid (AA) and dihomo gamma linolenic acid (DGLA) are the predominant LCPUFA produced from omega 6 LA. Eicosapentanoic acid (EPA) and Decosahexanoic acid (DHA) are the predominant LCPUFAs produced from omega 3 ALA. Omega 6 LCPUFA Arachidonic acid is synthesized in the body from LA or obtained from food of animal origin meat, egg and milk. Omega 3 LCPUFAs, EPA and DHA are synthesized in the body from ALA or obtained from cold water fish or algae and food of animal origin. Genetically modified fungi are also used for commercial production of omega 6 AA and omega 3 EPA and DHA. Omega 6 and 3 LCPUFAs are incorporated into the lipid bilayer as esters of phospholipids of the cell membrane and released according to the stimuli and get metabolized to ecosanoids and decosanoids, which have hormone like activity in cell signaling.

Ecosanoids are signaling molecules made by oxidation of 20 carbon PUFAs of omega 3 or omega 6 series. They exert complex control over many bodily systems, mainly inflammation, immunity and as messengers in the central nervous system. The network of controls that depends upon ecosanoids is the most complex in the human body. In general omega 6 ecosanoids are pro inflammatory and omega 3 ecosanoids are anti-inflammatory. The amount of balance of these fats in a person's diet will affect the body's ecosanoids controlled functions, with effects on cardio vascular diseases, cancer, blood pressure, arthritis, asthma, allergy, psychiatric diseases, recovery from infections and healing. Many drugs presently in use, like anti-inflammatory aspirin and other NSAID, anti-allergic drugs, anti-acidity drugs, psychiatric medication etc. act by modulating this ecosanoids pathway.

Unlike the other organs of the body, brain and nervous system has high content of fat. 60% of the dry weight of brain is fat and Decosahexanoic acid (DHA, C22, 6 n-3) an omega 3 LCPUFA is a primary structural component of human brain, retina, sperm, testicles and skin. DHA comprises 40% of PUFAs in brain, 60% PUFAs in retina and 50% of the weight of a neuron's plasma membrane. DHA modulates the carrier-mediated transport of choline, glycine, and taurine; the function of delayed rectifier potassium channels, and the response of rhodopsin contained in the synaptic vesicles, among other functions.

Human beings are evolved on a diet with a ratio of omega 6 to omega 3 essential fatty acids (EFA) of approximately 1:1, whereas in modern diets the ratio has increased to 10-40:1. Modern diet is deficient in omega 3 fatty acids, and has excessive amounts of omega 6 fatty acids, compared with the diet on which human beings evolved and their genetic patterns were established. Inside the body these two series of fatty acids omega 6—LA, GLA, DGLA, AA and its metabolites ecosanoids and omega 3—ALA, EPA, DPA, DHA and its metabolites ecosanoids and decosanoids follow parallel pathways, continually competing with each other for chemical conversion to various structures and molecules inside and outside the cells. An excess of dietary omega 6 compared to omega 3 fatty acids as in the modern processed food results in over production of omega 6 Arachidonic acid and its incorporation on the lipid bilayer leads to an increased release of AA with a parallel decrease in release of anti-inflammatory EPA during exposure to allergens and pollutants which is also increased due to industrialization and this leads to synthesis of a series of prostaglandins, prostacyclins, thromboxans, leucotrins and lipoxins which are pro inflammatory in nature. They are involved in the pathogenesis of modern DRNCD (diet related non communicable diseases) or the so called life style diseases like cardio vascular diseases, cancer, immune and inflammatory diseases, behavioral abnormality, asthma, allergy, arthritis etc. The deficiency of omega 3 fatty acids in the diet leads to its deficiency in the lipid bilayer and the omega 3 series ecosanoids which has an anti-inflammatory activity. Omega 3 EPA series of ecosanoids, resolvins and DHA series resolvins, protectants and maresins are anti-inflammatory and protective in function. Therefore maintaining a balance in consumption of these two fatty acids omega 6 and omega 3 fatty acids in the diet is essential for optimal growth, development, repair, maintenance of health, recovery from diseases, and healing of wounds.

TABLE 1
Major types of omega 6 fatty acids and their functions

| Type of omega 6 FAs | Functions |
|---|---|
| Linoleic acid (LA) | Essential fatty acid for the synthesis of GLA, |
| 18:2 n-6 | DGLA and AA |
| | Part of cell membrane lipid bilayer |
| Gama Linolenic acid | Semi essential fatty acid |
| (GLA) | Can be synthesized by the body from LA |
| 18:3 n-6 | Part of cell membrane lipid bilayer |
| | Used for the synthesis of DGLA and AA |
| Dihomo Gamma | Semi essential fatty acid |
| Linoleic acid DGLA | Can be synthesized by the body from LA and |
| 20:3 n-6 | GLA |
| | Part of cell membrane lipid bilayer |
| | Synthesis of DGLA series Ecosanoids |
| Arachidonic acid AA | Conditionally essential fatty acid - infants |
| 20:4 n-6 | Can be synthesized by the body from LA, GLA |
| | and DGLA |
| | Part of cell membrane lipid bilayer |
| | Synthesis of AA series ecosanoids |

TABLE 2
Major types of omega 3 fatty acids and their functions

| Type of omega 3 | |
|---|---|
| Fatty acid | Functions |
| Alfa Linoleic acid | Essential fatty acid for the synthesis of EPA, DHA |
| ALA 18:3 n-3 | Part of cell membrane lipid bilayer |
| Ecosapentanoic acid | Conditionally essential fatty acid - infants |
| EPA 20:5n-3 | Can be synthesized by the body from ALA |
| | Used for the synthesis of EPA series ecosanoids |
| | Used for the synthesis of DHA |
| | Part of cell membrane lipid bilayer |
| DecosaHexanoic | Conditionally essential fatty acid - infants |
| acid DHA 22:6 n-3 | Can be synthesized by the body from essential |
| | fatty acid ALA |
| | Major component of nervous system, retina, skin, |
| | testicles and sperm |
| | Production of Decosanoids |

TABLE 3
Type of omega 6 and 3 fatty acids with omega 6 to 3 ratio in
common cooking oils

| Sl. No. | Oil/Fat | Omega 6 FAs | Omega 3 FAs | Omega 6:3 |
|---|---|---|---|---|
| 1 | Coconut Oil | Linoleic | ~ | 2:0 |
| | | Acid (LA) | | |
| 2 | Palm Oil | Linoleic | ~ | 10:0 |
| | | Acid (LA) | | |
| 3 | Sunflower Oil | Linoleic | ~ | 66:0 |
| | | Acid (LA) | | |
| 4 | Safflower Oil | Linoleic | ~ | 74:0 |
| | | Acid (LA) | | |
| 5 | Corn Oil | Linoleic | ~ | 57:0 |
| | | Acid (LA) | | |
| 6 | Cotton Seed | Linoleic | ~ | 51:0 |
| | Oil | Acid (LA) | | |
| 7 | Gingili Oil | Linoleic | ~ | 45:0 |
| | | Acid (LA) | | |
| 8 | Ground nut Oil | Linoleic | ~ | 30:0 |
| | | Acid (LA) | | |
| 9 | Rice Bran Oil | Linoleic | Alfa Linolenic | 24:1 |
| | | Acid (LA) | Acid (ALA) | |
| 10 | Olive Oil | Linoleic | Alfa Linolenic | 13:1 |
| | | Acid (LA) | Acid (ALA) | |
| 11 | Butter/Ghee | Linoleic | Alfa Linolenic | 10:1 |
| | | Acid (LA) | Acid (ALA) | |
| 12 | Lard (Pig) | Linoleic | Alfa Linolenic | 10:1 |
| | | Acid (LA) | Acid (ALA) | |
| 13 | Tallow | Linoleic | Alfa Linolenic | 10:1 |
| | | Acid (LA) | Acid (ALA) | |
| 14 | Soya bean Oil | Linoleic | Alfa Linolenic | 7:1 |
| | | Acid (LA) | Acid (ALA) | |
| 15 | Walnut Oil | Linoleic | Alfa Linolenic | 5:1 |
| | | Acid (LA) | Acid (ALA) | |
| 16 | Mustard/Rape | Linoleic Acid | Alfa Linolenic | 2.5:1 |
| | seed oil | (LA) | Acid (ALA) | |
| 17 | Canola Oil | Linoleic Acid | Alfa Linolenic | 2.2:1 |
| | | (LA) | Acid (ALA) | |

TABLE 4
Type of omega 6 and 3 FAs and omega 3, 6 ratio in food
supplement oils

| Sl. | Food | | | Omega 6:3 |
|---|---|---|---|---|
| No. | Supplement | Omega 6 FAs | Omega 3 FAs | ratio |
| 1 | Cod-liver Oil | LA | EPA, DHA | 0.1:2 |
| 2 | Salmon Oil | LA | EPA, DHA | 0.1:2 |
| 3 | Sardine Oil | LA | EPA, DHA | 0.1:1 |
| 4 | Flax Seed Oil | LA | ALA | 0.3:1 |
| 5 | Evening | LA, GLA | ALA | 85:0.4 |
| | primrose Oil | | | |
| 6 | Borage Seed oil | LA, GLA | ~ | 58:0 |

TABLE 5
five different sample groups of Ingredients for production of
optimized nutrient oil with LA as omega 6 and ALA as omega 3

| | | | |
|---|---|---|---|
| 1 | Sunflower Seed | Flax seed | Gingili Seed |
| 2 | Soya Beans | Perilla Seed | Olive |
| 3 | Perilla Seed | Safflower Seed | Avocado |
| 4 | Flax seed | Walnut | Garden Cress Seed |
| 5 | Corn Seed | Sacha Inchi | Macadamia Seed |

TABLE 6
Five different sample groups of Ingredients for production of
optimized nutrient oil with LA as omega 6 and ALA + EPA + DHA as
omega3

| | | | | |
|---|---|---|---|---|
| 1 | Sunflower Seed | Flax seed | Chia Seed | Algae oil |
| 2 | Soya Beans | Perilla Seed | Garden Cress | Salmon oil |
| 3 | Perilla Seed | Safflower Seed | Avocado | Krill oil |
| 4 | Flax seed | Walnut | Gingili Seed | Sardine oil |
| 5 | Corn Seed | Sacha Inchi | Sea Buckthorn | Squid oil |

TABLE 7
Five different sample groups of Ingredients for the production of
optimized nutrient oil with LA + GLA as omega 6 and ALA as omega 3

| | | | | |
|---|---|---|---|---|
| 1 | Sunflower Seed | Flax seed | Gingili Seed | Evening |
| | | | | Primrose |
| 2 | Soya Beans | Perilla Seed | Olive | Black current |
| 3 | Perilla Seed | Watermelon | Avocado | High GLA |
| | | Seed | | Safflower |
| 4 | Flax seed | Walnut | Garden cress | Borage Seed |
| 5 | Corn Seed | Sacha Inchi | Macadamia | Evening Prim |
| | | | | rose |

TABLE 8
Five different sample groups of Ingredients for production of
optimized nutrient oil with LA + GLA as omega 6 and
ALA + EPA + DHA as omega 3

| | | | | | |
|---|---|---|---|---|---|
| 1 | Sunflower | Flax seed | Gingili Seed | Evening | Algae oil |
| | Seed | | | Primrose | |
| 2 | Soya Beans | Perilla Seed | Olive | Black | Salmon |
| | | | | current seed | Oil |
| 3 | Perilla Seed | Rice Bran | Hemp seed | High GLA | Krill oil |
| | | | | Safflower | |
| 4 | Flax seed | Cotton | Garden | Borage | Sardine oil |
| | | Seed | cress | Seed | |
| 5 | Corn Seed | Sacha Inchi | Macadamia | Evening | Squid Oil |
| | | | | primrose | |

TABLE 9
Five different sample groups of Ingredients for production of
optimized nutrient oil with LA + AA as omega 6 and
ALA + EPA + DHA as omega 3

| | | | | | |
|---|---|---|---|---|---|
| 1 | Sunflower Seed | Flax seed | Gingili | Algae oil | Fungus |
| | | | Seed | | Mortierellaalpina |
| 2 | Soya | Perilla Seed | Chia Seed | Salmon Oil | Fungus |
| | Beans | | | | Mortierellaalpina |
| 3 | Perilla | Safflower | Avocado | Krill oil | Fungus |
| | Seed | Seed | | | Mortierellaalpina |
| 4 | Flax seed | Walnut | Garden | Sardine oil | Fungus |
| | | | cress | | Mortierella |
| | | | | | alpine |
| 5 | Corn Seed | Sacha Inchi | Macadamia | Fungi oil | Fungus |
| | | | | (recombinant) | Mortierellaalpina |

TABLE 10
Five different sample groups of Ingredients for production
of optimized nutrient oil with LA + GLA +
AA as omega 6 and ALA + EPA + DPA + DHA as omega 3

| | | | | | | |
|---|---|---|---|---|---|---|
| 1 | Sun- | Flax | Gingili | Evening | Algae | Fungus |
| | flower | seed | Seed | Prim- | oil | Mortierella |
| | Seed | | | rose | | alpina |
| 2 | Soya | Perilla | Chia Seed | Black | Salmon | Micro |
| | Beans | Seed | | current | Oil | algae |
| | | | | seed | | |
| 3 | Perilla | Cotton | Avocado | High | Krill | Fungus |
| | Seed | Seed | | GLA | oil | Mortierella |
| | | | | Saf- | | alpine |
| | | | | flower | | |
| 4 | Flax | Walnut | Garden | Borage | Micro | Fungus |
| | seed | | cress | Seed | algae | Mortierella |
| | | | | | | alpine |
| 5 | Corn | Sacha | Macadamia | Evening | Fungi | Fungus |
| | Seed | Inchi | | prim- | oil | Mortierella |
| | | | | rose | (Recom- | alpina |
| | | | | | binant) | |

Table 1 shows major type of omega 6 fatty acids and their functions. Table 2 shows major type of omega 3 fatty acids and their functions. Table 3 gives details of type of omega 6 and 3 fatty acids with omega 6 to 3 ratio in common cooking oils. Table 4 gives details on type of omega 6 and 3 FAs and omega 3, 6 ratio in food supplement oils. From table 3 one can understand that most of the cooking oil currently on use has only negligible quantity or no omega 3 fatty acid, a good number of them have high omega 6 content. If one uses sun flower oil, safflower oil, corn oil, cotton seed oil, partially hydrogenated oil, rice bran oil, gingili oil, ground nut oil etc. for cooking, baking and frying; the omega 6, 3 ratios in the diet may go up around 10-70:1.

The cooking oils which provide reasonably high omega 3 are mustard oil and canola oil. Mustard oil contain about 6% omega 3 and omega 6:3 ratio is 2.5:1, but it has a very high concentration of erusic acid, which in large quantity can damage heart and outweighs the benefit of its omega 3 content. Canola was developed through traditional plant breeding from rape seed which has low erusic acid content and 10% omega 3 with omega 6, 3 ratio of 2.2:1. Soya bean oil contains 6.5% omega 3 but, it has high content of omega 6, so the ratio is high-7:1. Walnut oil contains 10% omega 3 but, the omega 6 is high with ratio 5:1. The currently available cooking oils have omega 6, 3 ratios ranging from 74:0 to 2.2:1. Hydrogenated vegetable oil like vanaspathi, dalda, bakery shortening etc. have high trans-fat and omega 6 content and no omega 3 and emerging scientific data shows the possible negative effects on health. Cod liver oil has high content of vitamin A and vitamin D and if taken more than 4 ml/day can lead to hyper vitaminosis and related complications. The marine oils including cod liver oil, sardine oil, salmon oil, krill oil, squid oil, algae oil contain LCPUFA; omega 3 EPA and DHA with a very low content of omega 6 LA and AA, if taken in large quantity for long time it can leads to imbalance in the essential fatty acid metabolism and body functions, especially blood clotting mechanism leading to bleeding complications.

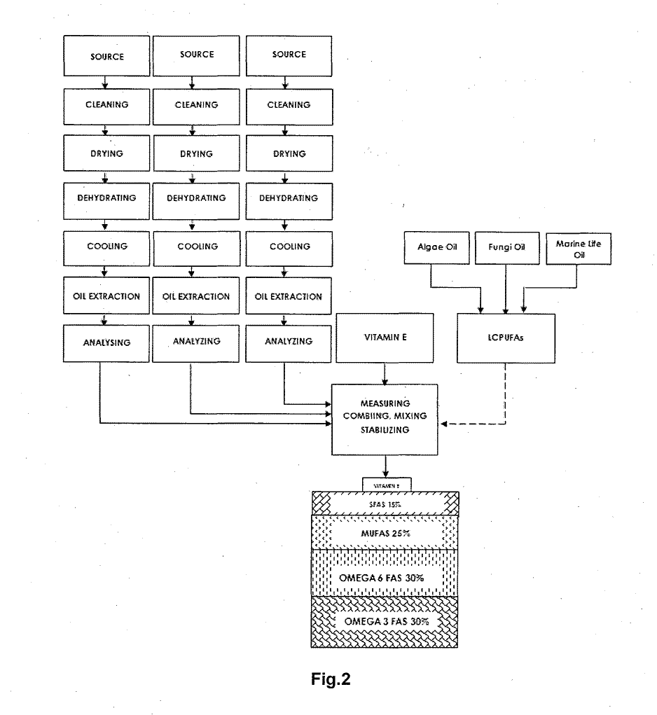

FIG. 2 shows a flow diagram depicting the various stages in the method of production of the optimized nutrient oil having optimized nutrient fatty acid composition according to an exemplary embodiment under the invention. The preferred embodiment of our invention is a fatty acid composition with omega 6 to 3 ratio 1:1 to 1:1.25. In the active phase of inflammation or those who are already on a high omega 6 diet it is better to have a slightly higher omega 3 like omega 6 to 3 ratio 1:1.25 to balance the inflammatory activities along with the modern drugs to control disease progress. The balancing of the omega 6 and 3 fatty acids at a cellular level, that is in the cell plasma membrane lipid bilayer will optimize the release of omega 6 and omega 3LCPUFAs from the plasma membrane and the synthesis of ecosanoids and decosanoids in response to stimuli including allergens, pollutants, microbes and injury. Once the active inflammatory phase is over and the optimal essential/semi essential fatty acid level is achieved it is optimal to have omega 6 and 3 ratio in diet as 1:1.

The essential fatty acids with multiple unsaturated double bond are highly unstable and prone for lipid peroxidation on storage, after absorption in to the body and after incorporation in to the cell membrane. In vivo the free radical activation and lipid peroxidation and resultant cell damage can have deleterious effects on the body. To overcome this possibility and resultant problems in the preferred embodiment of our invention; stabilization of the fatty acid composition was done by adding vitamin E. The amount of vitamin E needed to stabilize a fatty acid composition depends on the total number of unsaturated double bonds in the composition. For calculation of the amount of unsaturated double bonds and the vitamin E needed is determined based on the formula (% R1×1+% R2×2+% R3×3+% R4×4+% R5×5+% R6×6) divided by the factor F. Where % R1 is the percentage of fatty acid with one double bond on the fatty acid chain in the composition, % R2 is the percentage of fatty acid with two double bonds on the fatty acid chain in the composition, % R3 is the percentage of fatty acid with three double bonds on the fatty acid chain in the composition, % R4 is the percentage of fatty acid with four double bonds on the fatty acid chain in the composition, % R5 is the percentage of fatty acid with five double bonds on the fatty acid chain in the composition, % R6 is the percentage of fatty acid with six double bonds on the fatty acid chain in the composition. The factor F is an arbitrary number 150.

Mechanization in different aspects of modern life and sedentary life brought a new challenge in the health care sector in the form of overweight and obesity which leads to many health hazards. For a calorie restricted diet in obesity and sedentary life reduction in dietary fat can be achieved only by providing a fatty acid composition with high essential fatty acids. In another preferred embodiment of the invention 60% (50-70%) of the fatty acids in the composition is essential or semi essential fatty acid with omega 6 to 3 ratio 1:1 to 1:1.25 so that one can reduce the calorie intake from fat in the diet. Body can easily synthesize saturated fat from starch. Monounsaturated fatty acid oleic acid is needed for plasma membrane synthesis. Even though body can synthesize monounsaturated fat from saturated fat elongation of the chain and incorporation of unsaturated bond needs considerable effort to the body. To reduce the stress on the body in another embodiment of the invention 25% (20-30%) of the fatty acids in the composition is monounsaturated fatty acid and 15% (10-20%) is saturated fatty acid.

Omega 6 LCPUFA arachidonic acid (AA) and omega 3 LCPUFA decosahexanoic acid (DHA) are needed for the development of nervous system and retina of embryos and fetus in uterus. This optimized nutrient fatty acid composition with 50-70% omega 3 and 6 fatty acid in 1:1 ratio will ensure balanced production of these two fatty acids in pregnant mothers and growing children there by an ideal supplement for pregnant and lactating mothers and growing children to ensure cognitive and eye sight development.

According to the preferred embodiment of the optimized nutrient fatty acid composition 25-35% of the total fatty acids as omega 3 fatty acid and 25-35% omega 6 fatty acid, wherein omega 6, 3 ratio 1:1 to 1:1.25% with total essential or semi essential fatty acid content 50-70%, 20-30% monounsaturated fatty acid and 10-20% saturated fatty acid which is stabilized and protected from lipid peroxidation in vitro and in vivo using vitamin E 0.8-4 mg/ml of the composition calculated using the formula (% R1×1+% R2×2+% R3×3+% R4×4+% R5×5+R6×6) divided by the factor F. For example in an optimized fatty acid composition containing 30% LA with 2 double bonds, 30% ALA with 3 double bonds, 25% oleic acid with one double bond and the remaining as saturated fat and non-fatty acid components without unsaturated double bonds; the formula will be (25×1+30×2+30×3) divided by 150=175 divided by 150=1.17. That means 1.17 mg of vitamin E has to be added to each milliliter of optimized nutrient fatty acid composition. Vitamin E is an essential nutrient needed for the body whose main function in the body is to act as an antioxidant and free radical scavenger. This vitamin E along with drying and dehydration of the raw materials before oil extraction, air tight/nitrogen filled packing and preventing light exposure on storage gives a safe way for improving the shelf life and preventing lipid peroxidation of the optimized fatty acid composition. If LCPUFAs including GLA (3 double bonds), AA (4 double bonds), EPA (5 double bonds) and DHA (6 double bonds) are used calculation is done accordingly and the needed quantity of vitamin E will be more.

This optimized nutrient fatty acid composition can be used as a food, a food ingredient, a food supplement, a cooking oil, a skin care oil, a hair care oil, a carrier oil for nutrients and drugs, a base oil for ointments, creams and lotions for cosmetic and pharmaceutical preparations and a base oil for preparation of intravenous lipid emulsion. Using this oil with high essential fatty acid and optimal omega 3, 6 FAs ratio and stabilized with antioxidant vitamin E can result in optimization of bodily functions, maintenance of health, prevention of disease, promotion of growth, healing and recovery, and delay aging. Optimized nutrient fatty acid composition with lower concentration of omega band 3 fatty acids can also be prepared either by selecting the source with low omega 6 and 3 fat and high monounsaturated/saturated fat or by adding fully hydrogenated fat without altering the omega 6 to 3 ratio 1:1 to 1:1.25.

The optimized nutrient oil having optimized nutrient fatty acid composition wherein ALA as omega 3 FA and LA and GLA as omega 6 and omega 6, 3 ratio 1:1 to 1:1.25 can be made from edible seeds and nuts. If semi essential omega 3 fatty acids EPA and DHA are to be included, algae or marine life source is used. If semi essential omega 6 GLA is included special seeds like borage seed, high GLA safflower seed, hemp seed, evening primrose seed, black current seed etc. are used. AA is sourced from the fungus Mortierella alpina.

In an exemplary embodiment under the invention process steps involved in the production of optimized nutrient fatty acid composition is shown in FIG. 2. The source of omega 3 fatty acid ALA include canola, walnut, flax seed, perilla, garden cress, hemp seed, sachainchi, hazel nut, chia seeds, mustard seed, and clary sage. The source of omega 3 LCPUFAs—EPA and DHA include marine algae, cold water marine life including fish oil, krill oil, squid oil and fungi oil are used. The source of omega 6 fatty acid LA include safflower seeds, sunflower seeds, corn, cotton seed, soya beans, rice bran, ground nut, water melon seed, cashew, almond, gingili, pumpkin seed, and poppy seed. The source of semi essential GLA include high GLA safflower seed, borage seed, evening primrose seed, black current seed, and hemp seed. When long chain omega 6 AA is needed fungus Mortierella alpina is used. The source of monounsaturated fatty acids include olive, sesame (gingili), palm oil, ground nut, rice bran, avocado, mustard, lard, tallow, macadamia seed, and sea buckthorn. Most common MUFA is oleic acid but macadamia and sea buck thorn contains palmitoleic acid, and mustard contains erusic acid. The source of saturated fat includes coconut, palm oil, butter, ghee, lard, tallow and fully hydrogenated vegetable oil. With advancement of biotechnology genetically modified fungi are used for commercial production of LCPUFAs including AA, EPA and DHA. Such omega 6 and 3 LCPUFAs also can be used for getting the desired composition of omega 6 and 3 fatty acids. Microalgae grown in bioreactors can also be used for the production of LCPUFAs.

The preferred embodiment of method of preparation of said optimized fatty acid composition include selecting the source of fatty acids from a group including plant source, genetically modified plant source, animal source, genetically modified animal source, marine life source, genetically modified marine life source, algal source, genetically modified algal source, fungal source, genetically modified fungal source, microorganism, and genetically modified microorganism. Oil extraction is done after cleaning, drying and dehydrating the selected raw materials using known technologies for the selected source materials. After cleaning and air drying, dehydration is done at 40-90 degree centigrade to remove water content thereby protecting the oil from hydrolytic rancidity and improve the stability of the composition. Sample of each oil is analyzed to know the exact fatty acid composition of respective source. Selected oils based on the fatty acid analysis data is measured and combined to get the preferred concentration of omega 3 fatty acid, omega 6 fatty acid, MUFAs and SAFs with omega 6 to 3 ratio 1:1 to 1:1.25. Vitamin E is added for preventing lipid peroxidation of the unsaturated fatty acids in vitro and in vivo. When adding vitamin E different factors like proportion of unsaturated fatty acids, the degree of unsaturation, the position of unsaturated bond in the fatty acids chain are considered to decide the quantity of vitamin E. For example omega 3 fatty acid DHA containing 6 double bonds are more unstable and vulnerable for lipid peroxidation and need more vitamin E to stabilize DHA. The amount of vitamin E need for stabilizing the composition is determined by the formula (% R1×1+% R2×2+% R3×3+% R4×4+% R5×5+% R6×6) divided by the factor F, wherein % R1 is the percentage of fatty acid with one double bond in the fatty acid chain, % R2 is the percentage of fatty acid with two double bonds in the fatty acid chain, % R3 is the percentage of fatty acid with three double bonds in the fatty acid chain, % R4 is the percentage of fatty acid with four double bonds in the fatty acid chain, % R5 is, the percentage of fatty acid with five double bonds in the fatty acid chain, % R6 is the percentage of fatty acid with six double bonds in the fatty acid chain, the factor F is an arbitrary number 150.

Slow mixing is done at RPM 200 to 500 for 2 to 5 minutes in a stirrer. Sample is taken and analyzed to ensure the preferred composition and if needed finer adjustments are done. Packaging is done immediately in air tight/nitrogen filled and opaque containers to avoid contact with oxygen and exposure to light. Oil extraction can be done from different sources separately or after mixing the sources in special proportion to get the oil of omega 6 FAs, Omega 3 FAs, MUFAs and SFAs in the desired ratio.

Dehydration before oil extraction, avoiding exposure to light and oxygen and addition of antioxidant vitamin E in sufficient quantity makes the oil stable, prevents rancidity and prolongs shelf life. Vitamin E is a fat soluble vitamin and it is absorbed into the body along with the fatty acids. Inside the body also vitamin E protects the fatty acids especially the LCPUFAs incorporated in the lipid bilayer of the cell membrane from oxidative damage there by prevent cell damage and aging.

Selection of seeds and the quantity of each seed depends on the oil content of the seed and the fatty acid profile of the seed oil. Different combinations of the seeds can be used according to the seasonal and geographical availability. Most of the seeds and nuts are cultivated and available in Indian markets. Some seeds like Sacha Inchi, Chia Seed, Macadamia, High GLA safflower, Borage seed etc. are not cultivated in India but cultivated and used as food or food supplement in other countries. Five different examples of selection of ingredients for the production of optimized nutrient oil having different fatty acid composition of varying types of omega 6 and 3 fatty acids, but omega 6, 3 ratio 1:1 to 1:1.25 with total omega 6 and 3 FAs content 60% (50-70%) is given in tables 5-10. Table 5 shows five different sample groups of Ingredients for production of optimized nutrient oil with LA as omega 6 and ALA as omega 3. Table 6 shows five different sample groups of Ingredients for production of optimized nutrient oil with LA as omega 6 and ALA+EPA+DHA as omega3. Table 7 shows five different sample groups of Ingredients for the production of optimized nutrient oil with LA+GLA as omega 6 and ALA as omega 3. Table 8 shows five different sample groups of Ingredients for production of optimized nutrient oil with LA+GLA as omega 6 and ALA+EPA+DHA as omega 3. Table 9 shows five different sample groups of Ingredients for production of optimized nutrient oil with LA+AA as omega 6 and ALA+EPA+DHA as omega 3. Table 10 shows five different sample groups of Ingredients for production of optimized nutrient oil with LA+GLA+AA as omega 6 and ALA+EPA+DPA+DHA as omega 3. DHA an omega 3 fatty acid is needed for the development of brain, nervous system and retina of the developing fetus. The use of this optimized nutrient oil rich in omega 3 by pregnant women will improve brain and retinal development in fetus, the use in lactating mother will improve the DHA level of breast milk and help brain and cognitive growth of the baby. AA, EPA and DHA are conditionally essential fatty acids and to be added in infant formula.

This optimized fatty acid composition with omega 6 and 3 FAs with omega 3, 6 ratio 1:1 to 1:1.25 is helpful for prevention and treatment of inflammation and immune related diseases like cardiovascular diseases, cancer, dyslipidemia, hypertension, insulin resistance and type II diabetes, autoimmune disease like thyroiditis, arthritis, ulcerative colitis, asthma, allergy psychiatric disorders etc. because the pathogenesis of these diseases have a base in over expression of ecosanoids derivative of omega 6 arachidonic acid (AA) which in turn is produced from LA. A balance of omega 6 AA and omega 3 EPA is essential to keep away these so called diseases of civilization or life style diseases which can be achieved by using optimized nutrient fatty acid composition. For achieving optimal growth body should get enough essential fatty acids from the diet, in a balanced ratio which will optimize the body functions and there by optimize growth and development in children and repair, recovery and health in adults. It can be taken as a food ingredient or as food supplement directly or as capsules.

Different type of omega 3 fatty acids like Aalpha Linoleic Acid (ALA), Steriodonic Acid (SDA), Ecosatetraenoic Acid (ETA), Ecosapentanoic Acid (EPA), DecosaPentaenoic Acid, and DecosaHexanoic acid (DHA); omega 6 fatty acids like Linoleic acid (LA), Gama Linoleic Acid (GLA), Dihomo Gama Linoleic Acid (DGLA) and Arachidonic Acid (AA) and monounsaturated fatty acids like (MUFA) oleic, palmitoic acid, vaccenic acid and erucic acid can be used in the composition. Saturated fatty acids can be selected from short chain SFAs, medium chain SFAs, and long chain SFAs. Fatty acids can be selected from different sources like plant source, genetically modified plant source, animal source, genetically modified animal source, marine life source, genetically modified marine life source, algal source, genetically modified algal source, fungal source, genetically modified fungal source, microorganism, and genetically modified microorganism.

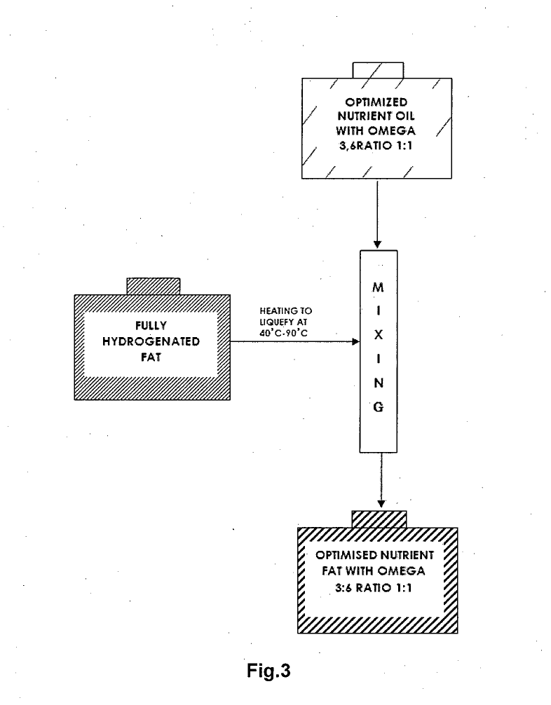

The flow diagram FIG. 3 describes another embodiment of the invention, how optimized fat of consistency of butter/spread is prepared from optimized nutrient fatty acid composition. Fully hydrogenated fat is hard, and is not comfortable to use in cooking. Combining Optimized nutrient oil with fully hydrogenated trans-fat free fat in suitable proportion can yield highly nutritious healthy fat with omega 3, 6 ratio 1:1 with the consistency of butter, dalda/vanaspathi which can be used comfortably for cooking, baking or as spread.

This optimized fat with optimal omega 3 to 6 ratio can be mixed with food ingredients including milk solids, water, emulsifiers, natural/synthetic flavors and colors for the production of cream, margarine, mayonnaise, bread spread etc. of varying consistency and taste. Fully hydrogenated oil is hard at room temperature. It is heated at 40-90 degree centigrade to liquefy and mixed with optimized nutrient oil in suitable proportion to get fat solids or semi solids of desired consistency. Further this optimized fat can be mixed with water and emulsifiers like soya lecithin, egg lecithin etc. to make emulsions of varying consistency according to the percentage of water, emulsifiers and optimized nutrient oil used. Further addition of milk solids will give cream with omega 3, 6 ratio 1:1. Optimized nutrient fat of suitable consistency can be used for production of margarine, mayonnaise, spreads etc. with omega 3, 6 ratio 1:1 by adding suitable spices, nutrients, coloring and flavoring agents as per the standard practices in food industry which opens up the possibility of a wide range of healthy food products with essential fat and omega 3, 6 ratio 1:1.

Currently available cosmetic and pharmaceutical topical preparations oils, ointments, creams and lotions are commonly based on petroleum byproducts which have no nutritive value and their absorption into the body is not desirable. Mineral oil is a colorless, odorless, light mixture of alkenes in the C15 to C49 range, a distillate of petroleum. It is a major ingredient in body care products, cosmetic products, pharmacological products, baby oil, hair care oil etc. Low cost and long shelf life make it favorite for pharmaceutical and cosmetic industry.

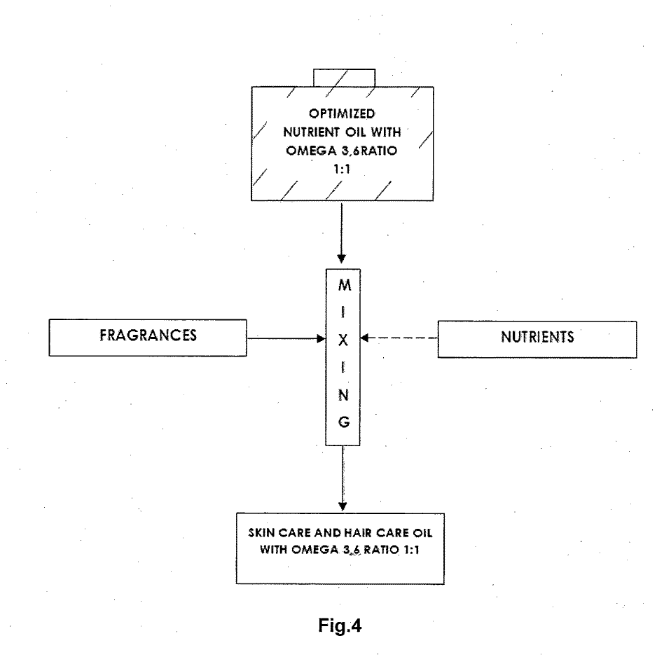

Another preferred embodiment of the invention is depicted in flow diagram FIG. 4 production of skin care and hair care preparation using optimized nutrient fatty acid composition for making body care oil suitable fragrances are added to the optimized nutrient oil and mixed in stirrer at RPM 200-500/minute. Natural fragrance of flowery, fruity, woody or leafy essential oil or artificial fragrances can be added to make it pleasant. Natural fragrant essential oil including rose, lavender, jasmine, ylangylang sandal wood, agar wood, cedar wood, orange, lime, apple, lemon grass, citronella, musk, eucalyptus with special smell and characters can be added or artificial fragrances may be used. So with optimized Nutrient oil opens up a wide range of body and hair care products which are nourishing, protective and pleasant. Essential oil is added to get desired fragrance. Either single or a mixture of different essential oils and, or artificial fragrances is used for the desired effect. Further optimized nutrient oil in suitable proportion can be used instead of or along with mineral or silicon oil for the production of creams and lotions for skin, hair care and in pharma production using standard production procedures.

While preparing optimized nutrient oil for making body care products omega 6 GLA and omega 3 EPA and DHA sources can be included for utilizing their special properties. This fragrant optimized oil can be used as body care oil, hair and nail care oil. When applied on the skin part is absorbed into the skin, and the left over oil due to high content of unsaturated double bonds on exposure to air and body heat form a thin layer over the skin. This film reflects light and gives radiance to the skin, and protects the skin from harmful rays of the sun, toxins and pollutants. Antioxidant vitamin E is absorbed into the skin layers and protects the cell membrane from oxidative damage and helps to maintain the youthfulness of skin. Soaps and detergents wash away the protective sebaceous secretions from the skin. This nutrient oil is ideal for application after bath. It moisturizes, nourishes and protects the skin, hair and nails.

ALA EPA, DHA and GLA has anti-inflammatory property which helps in recovery of dermal cells from sun damage and there by lightens the skin and helps to remove tanning. They improve the insulin sensitivity when applied directly on the skin which helps to reduce acanthosisnigricans associated with type II diabetes. ALA, GLA and LCPUFAs have inhibitory effect on 5 alpha reductase enzyme which converts testosterone to potent dihydrotestosterone (DHT). Over activity of 5 alpha reductase on hair follicle is the causative factor for abnormal hair loss or baldness in men and women. Optimized nutrient oil with ALA, GLA and LCPUFAs gives a natural remedy for androgen DHT dependent hair loss. Hirsutism or abnormal hair growth in females with PCOD also can be controlled with optimized nutrient oil with ALA, GLA & LCPUFAs due to its effects on improving insulin sensitivity and 5∝ reductase inhibition.

The optimized nutrient oil is nourishing and protective at the same time and can be used as a superior oil for body care and hair care and also for the preparation of lotions, creams and ointment for cosmetic and pharmaceutical use. Unsaturated fatty acids with its curving at the double bonds improves the absorption of nutrients and medicines along with the triglyceride absorption. When optimized nutrient fatty acid composition is regularly applied on the skin the poly unsaturated fatty acid content of the dermal cell membrane will be increased which improves the fluidity of the cell membrane and makes it more flexible and allow easy transport of nutrients, waste materials and drugs across the cell membrane. When optimized nutrient oil rich in unsaturated fatty acids and stabilized with vitamin E is applied on the skin more LCPUFAs are produced and incorporated on the lipid bilayer of the dermal cells and increases the fluidity of cell membrane while vitamin E protects the LCPUFAs from lipid peroxidation and thereby reduces the age related wrinkling of the skin.

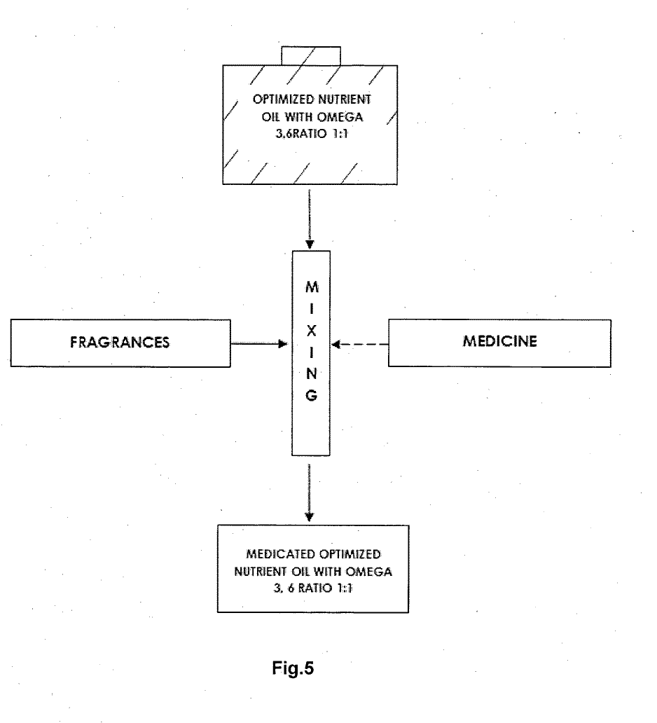

The optimized nutrient oil can be used as a media for delivery of nutrients and drugs through skin and mucous membrane. FIG. 5 shows another preferred embodiment of the invention, production of medicated preparations from optimized nutrient fatty acid composition. For production of medicated optimized nutrient oil for topical application the medicine is added to the optimized nutrient oil along with fragrances in specific proportions according to the desired effect and mixed well.

Essential fatty acids and LCPUFAs with the curving of the chain at the site of unsaturation increases the fluidity of the cell membrane lipid bilayer and eases the transfer of nutrients and drugs across the membrane. This quality can be utilized for local or systemic delivery of drugs applied topically to skin for cosmetic action and for disorders of skin and joints like eczema, dermatitis, psoriasis, arthritis etc. which opens up wide range of products for cosmetic and pharmaceutical use.

Unlike the regular mineral oil based topical pharmaceutical products the optimized nutrient fatty acid based topical medicines will be absorbed easily and more effectively because of the presence of PUFAs which improves the cell membrane fluidity. Many medications which are already used for treatment can be used more effectively with optimized nutrient oil.

Caffeine incorporated in optimized nutrient oil on dermal application lightens the skin by reducing melanin and removes cellulite by improving the microcirculation. Incorporation of methotrexate into optimized nutrient oil can be used for local delivery of the drug in the treatment of psoriasis and rheumatoid arthritis. Delivery of anti-inflammatory agents for aches and pains also can be done more effectively by incorporating them in to optimized nutrient oil rather than mineral oil based ointments. Likewise there is a wide range of drugs which can be delivered topically for dermatological conditions including hair loss (fenasteride, dutasteride, minoxidil), hirsutism (eflornithine) etc. and can minimize the dose and side effects associated with medication and improve efficacy. Incorporation of hormones including estrogen, progesterone and testosterone into optimized nutrient oil for HRT can deliver the hormones, systemically or locally like vagina and urethra and give the benefit of by passing the liver and first pass metabolism and related side effects.

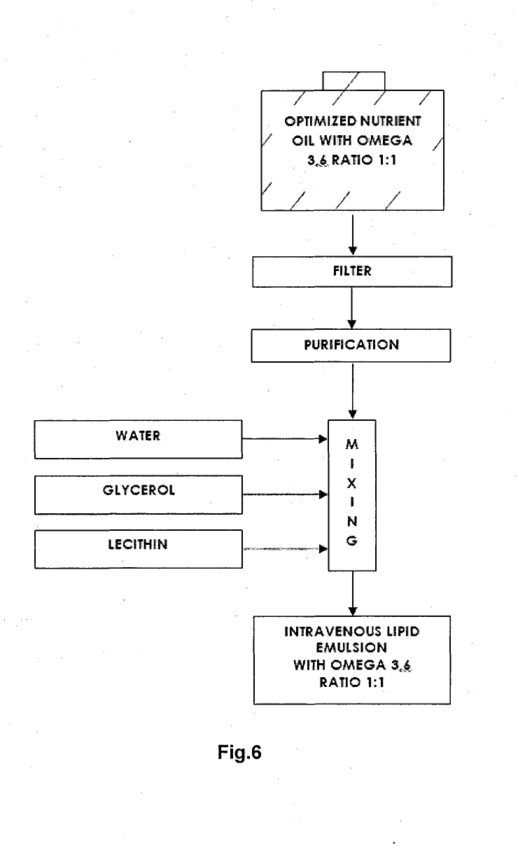

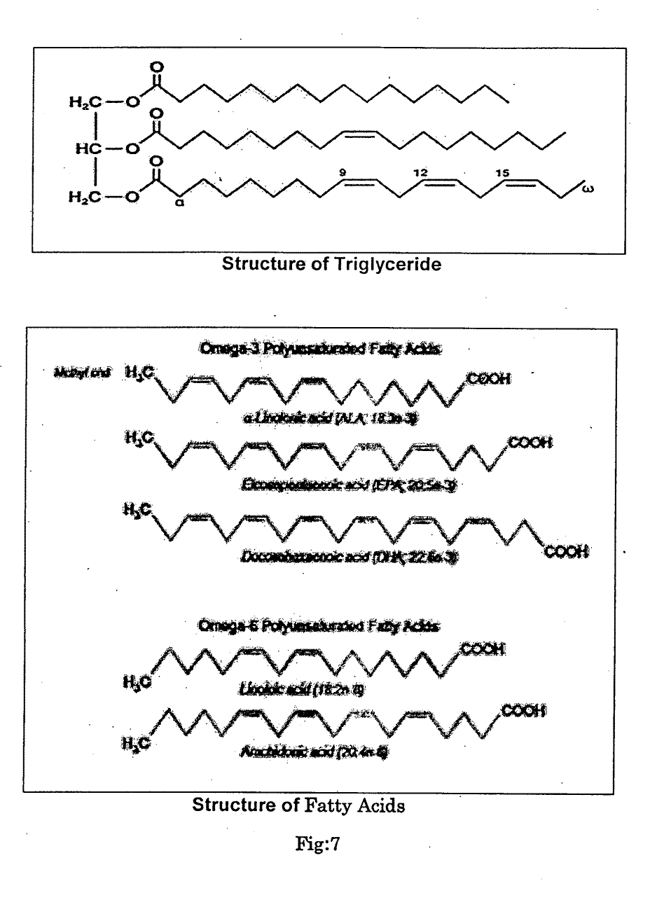

Another preferred embodiment of the invention is represented in FIG. 6 shows the preparation of intra venous lipid emulsion with omega 6, 3 ratio 1:1 to 1:1.25. For production of intra venous lipid emulsion with optimized nutrient oil, ultra-purification and filtration techniques are to be used for refining the oil. Then it is mixed with water and emulsifying agents' lecithin and glycerol in suitable proportion to make the lipid emulsion of desired concentration as in the standard pharmaceutical production techniques. The type of fatty acid selected depends on the end use, for example DHA and EPA is included in use for brain injury patients and DHA, EPA and AA are included in premature infant feeding.

Intra venous lipid emulsion is used for providing nutritional support for those who cannot take food orally and for premature infant feeding. It is also used for treatment of toxicity due to lipid soluble agents (example bupivacaine anesthetic drug). Another important use is to give intra venous lipid 3-5 minutes before cardiac reperfusion to prevent reperfusion injury in cardiology intensive care. The lipid emulsion containing optimized nutrient fatty acid composition will have better effects in treatments including in brain injury, before cardiac reperfusion, recurrent abortions, inflammatory disorders, and immune disorders.

Preparing intra venous lipid with this optimized nutrient oil will give more essential fatty acid with omega 3, 6 ratio 1:1 and vitamin E to prevent lipid per oxidation. Including omega 3 DHA the brain lipid will help in the recovery of patient with acute brain injuries. Including AA, EPA and GLA are also possible according to the need as in premature infant feeding. There are other newly emerging uses of intra venous lipid as in treatment of recurrent abortions. Here the use of this optimized nutrient oil having optimized nutrient fatty acid composition with omega 3, 6 ratio 1:1 will balance the ecosanoids production and thereby balance the immune and inflammatory reactions which will have crucial role in implantation and intra uterine growth of fetus.

### EXAMPLES

Oil was extracted from soybeans, flax seed, walnut, perilla seed, rice bran, sunflower seed, evening primrose seed, high GLA safflower seed, garden cress seed, sachainchi beans and green algae using conventional methods after cleaning, drying, and dehydration as shown in FIG. 2. Fatty acid analysis of the samples was done. Optimized nutrient fatty acid compositions were prepared using these oils.

### Example 1

4 liters of Soybean oil, 4.2 liters of flax seed oil, and 1.8 liters of perilla oil were measured and taken into a mixing vessel provided with stirrer to make a batch of 10 liters. 1.2 mg vitamin E was added for each ml of the optimized nutrient fatty acid composition. Slow mixing was done at 300 RPM for two minutes. Samples were taken for fatty acid composition analysis and found omega 3 fatty acid was around 32%, omega 6 fatty acid was 32% and monounsaturated fatty acids was 23% and 13% saturated fat including non-fatty acid components.

### Example 2

4.5 liters of Walnut oil, 5 liters of flax seed oil, and 0.5 liters of green algae oil were measured and taken into a mixing vessel provided with stirrer to make a batch of 10 liters. 1.3 mg vitamin E was added for each ml of the optimized nutrient fatty acid composition. Slow mixing was done at 300 RPM for two minutes. Samples were taken for fatty acid composition analysis and found omega 3 fatty acid was around 31%, omega 6 fatty acid was 30% and monounsaturated fatty acid was 25% and 14% saturated fat and non-fatty acid components.

### Example 3

2.4 liters of rice bran oil, 5.2 liters of perilla seed oil, 1.8 liters of evening primrose oil, and 0.6 liters of green algae oil were measured and taken into a mixing vessel provided with stirrer to make a batch of 10 liters. 1.6 mg vitamin E was added for each ml of the optimized nutrient fatty acid composition. Slow mixing was done at 300 RPM for two minutes. Samples were taken for fatty acid composition analysis and found omega 3 fatty acid was around 30%, omega 6 fatty acid was 30% and omega 9 was 23% and 17% saturated fat and non-fatty acid components.

### Example 4

4.5 liter of soybean oil, 5.2 liter of flax seed oil, 0.2 liter of green algae oil and 0.1 liter of rose essential oil were taken into a mixing vessel provided with stirrer to make a batch of 10 liters. 1.3 mg vitamin E was added for each ml of the optimized nutrient fatty acid composition. Slow mixing was done at 300 RPM for two minutes. Samples were taken for fatty acid composition analysis and found omega 3 fatty acid was 33%, omega 6 fatty acid was 32% and monounsaturated fatty acids was 23% and 12% saturated fat and non-fatty acid components.

### Example 5

3.7 liter of sunflower oil, 5.8 liter of flax seed oil, 0.4 liter of green algae oil, and 0.1 liter of agar wood essential oil were measured and taken into a mixing vessel provided with stirrer to make a batch of 10 liters. 1.3 mg vitamin E was added for each ml of the optimized nutrient fatty acid composition. Caffeine was added to get a concentration of 40 mg per ml. Enrichment of the oil with fat soluble vitamins vitamin A 350 mg/ml, vitamin D 60 mg/ml, vitamin K 10 mcg/ml was also done. Slow mixing was done at 300 RPM for two minutes. Samples were taken for fatty acid composition analysis and found omega 3 fatty acid was around 32%, omega 6 fatty acid was 32% and omega 9 was 22% and 14% saturated fat and non-fatty acid components.

### Example 6

3.8 liters of sachainchi oil, 4 liters of High GLA safflower oil, 2 liters of green algae oil and 0.2 liters of ylangylang essential oil were taken into a mixing vessel provided with stirrer to make a batch of 10 liters. 2.2 mg vitamin E was added for each ml of the optimized nutrient fatty acid composition. Slow mixing was done at 300 RPM for two minutes. Samples were taken for fatty acid composition analysis and found omega 3 fatty acid was 31%, omega 6 fatty acid was 29% and monounsaturated fatty acids was 25% and 15% saturated fat and non-fatty acid components.

### Example 7

4.3 liters of soybean oil, 5.5 liters of flax seed oil, and 0.2 liters of sandal wood essential oil were taken into a mixing vessel provided with stirrer to make a batch of 10 liters. 1.2 mg vitamin E was added for each ml of the optimized nutrient fatty acid composition. Estriol was added to get a concentration of 0.25 mg per ml. slow mixing was done at 300 RPM for two minutes. Samples were taken for fatty acid composition analysis and found omega 3 fatty acid was 32%, omega 6 fatty acid was 30% and monounsaturated fatty acids was 23% and 15% saturated fat and non-fatty acid components.

### Example 8

3.8 liters of soybean oil, 5 liters of flax seed oil, 1 liter of green algae oil, 0.2 liters of sandal wood essential oil were taken into a mixing vessel provided with stirrer to make a batch of 10 liters. 1.2 mg vitamin E was added for each ml of the optimized nutrient fatty acid composition. Slow mixing was done at 300 RPM for two minutes. Samples were taken for fatty acid composition analysis and found omega 3 fatty acid was 34%, omega 6 fatty acid was 32% and monounsaturated fatty acid was 22% and 12% saturated fat and non-fatty acid components.

### Example 9

3.8 liters of soybean oil, 5 liters of flax seed oil, 1 liter of green algae oil and 0.2 liter of sandal wood essential oil were taken into a mixing vessel provided with stirrer to make a batch of 10 liters. 1.2 mg vitamin E was added for each ml of the optimized nutrient fatty acid composition. Methotrexate was added to get a concentration of 0.5 mg per ml. slow mixing was done at 300 RPM for two minutes. Samples were taken for fatty acid composition analysis and found omega 3 fatty acid was 34%, omega 6 fatty acid was' 32% and monounsaturated fatty acids was 22% and 12% saturated fat and non-fatty acid components.

### Case Studies

The optimized nutrient fatty acid compositions produced as the embodiments of the present invention by the said method as described in Example 1 to 9 were administered to those who are needy and found to give encouraging results. Few of the relevant case studies are provided in the following examples which fully justify our contention that the surprising findings are attributable to synergetic effect of the constituent fatty acids in special proportion involved in the state of the art optimized nutrient fatty acid composition according to the present invention.

### Example 10

20 patients of age 25-35 yrs with anovulatory infertility due to polycystic ovarian disease were taken for study. Patients were divided into two groups. Both the groups were evaluated with routine urine test, blood test including hematology, biochemistry and hormone study. Both the groups were given standard modern medical treatment for induction of ovulation after correction of other fertility related problems. In addition the second group was asked to take 25 ml optimized nutrient oil prepared by a process as given in example-2 as cooking oil per day. The second group was also advised to avoid nuts and oil seeds and cooking oil containing omega 6 fatty acids to keep the omega 3 to 6 ratio around 1:1 in the food.

At the end of 3 months 5 patients out of 10 patients were pregnant in the first group and 8 patients out of 10 patients were pregnant in the second group. This showed the pregnancy rate was significantly higher in those who were on optimized nutrient oil compared to first group without the optimized nutrient oil. The quantity of ovulation induction drug used was also significantly lower in the second group who were taking optimized nutrient oil. The patients in the second group were asked to continue with the optimized nutrient oil throughout pregnancy. Pregnancy complications including miscarriage, pregnancy induced hypertension and premature birth were low in second group who were on optimized nutrient oil compared to first group.

The balanced ratio of omega 6 and 3 fatty acids in the diet of the second group with increased dietary intake of omega 3 with DHA than the ordinary diet of first group is the reason for the improved fertility and reduced pregnancy complications. Balanced 1:1 ratio of omega 3 to 6 promotes synthesis of longer chain omega 3 fatty acids EPA and DHA at cellular level. EPA has anti-inflammatory action and promotes implantation of embryos in the uterus and prevents preterm labor. DHA has crucial role in the development of brain and retina of the fetus.

### Example 11

10 patients of age 45-75 yrs with diabetes were taken for study. Patients were divided into two groups. Both the groups were evaluated with FBS, PPBS and HbA1C sensory receptor status study of foot using monofilament, tuning fork and vibretometer. Both the groups were given standard modern medical treatment for diabetes and diet advice. In addition, the second group was asked to take 25 ml per day optimized nutrient oil prepared by a process as given in example-1 as cooking oil and foot care oil prepared by a process as given in example 4 to massage on the hands and feet twice daily. The second group was also advised to avoid nuts and oil seeds and cooking oil containing omega 6 fatty acids to keep the omega 3 to 6 ratio around 1:1 in the food.

At the end of 2 months all the patients in the second group had better diabetes control as assessed by blood sugar and HbA1C. It was possible to reduce the drugs used for diabetic control in 3 patients out of 5 in the second group, but 2 out of 5 in the first group needed an increase in diabetic medication due to poor blood sugar control. Sensory receptor status study of foot using monofilament, tuning fork and vibretometer showed significant improvement in sensory perception in the second study group who were on optimized nutrient oil as food and foot care oil as example 1 & 4 respectively. In addition second group showed reduction in callus formation on the sole and reduction in wrinkles and aging signs of the skin especially on foot where the foot care oil was used.

The improvements in diabetic control and foot sensation of the second group who were on optimized nutrient oil as food and foot care oil was due to the omega 3 fatty acids activity as insulin sensitizer and control of inflammatory activity at a cellular level due to the balanced intake of omega 6 and 3 fatty acids in the food and also the fatty acids absorbed through skin. DHA an omega 3 fatty acid from algae oil is absorbed from the skin to the dermal cells including the sensory receptors and nerve cells which help in improvement in diabetic sensory neuropathy. The presence of long chain omega 3 fatty acids with more unsaturated double bonds make the cell membrane lipid bilayer more flexible and the resultant improved exchange of nutrients and waste material across the cell wall make the cell healthy and younger.

### Example 12

12 patients with hyperpigmentation on the face/neck (acanthosis nigricans)/under eye were taken for study.

Patients were divided into two groups. Both the groups were evaluated with physical check-up and laboratory tests including BRE, URE, blood sugar, vitamin D, LFT and RFT. Both the groups were given standard medical treatment for any abnormality detected in tests. In addition the second group was asked to take 25 ml per day optimized nutrient oil prepared by a process as given in example-1 as cooking oil and face and neck care oil prepared by a process as given in example 5 to apply on face and neck especially over the hyper pigmented area twice daily. The second group was also advised to avoid nuts, oil seeds and extra cooking oil in food to keep the omega 3 to 6 ratio around 1:1 in the food.

At the end of 30 days there was marked reduction in hyper pigmentation in the second optimized nutrient oil using group. But in the first group who were not on optimized oil at the end of study period either had an increase in pigmentation or the level of darkening remained same.

### Example 13

10 patients with male pattern hair loss (balding) were taken for study. Patients were divided into two groups. Both the groups were evaluated with physical check-up and laboratory tests including BRE, URE, blood sugar, LFT and RFT. Both the groups were given standard medical treatment for any abnormality detected in tests. In addition the second group was asked to take 25 ml per day optimized nutrient oil prepared by a process as given in example-3 as cooking oil and face and neck care oil prepared by a process as given in example 6 to apply on the scalp where hair loss was noted twice daily. The second group was also advised to avoid nuts, oil seeds and extra cooking oil in food to keep the omega 3 to 6 ratio around 1:1 in the food.

Patients were evaluated at the end of 30 days and 60 days. There was evidence of regrowth of hair as small hair at the area of hair loss in second optimized nutrient oil using group. But in the first group who were not on optimized oil at the end of study period either had an increase in hair loss or remained the same. Balance of omega 3 & 6 fatty acids as 1:1 with presence GLA& DHA inhibits the activity of 5 alpha reductase enzymes in the hair follicle which results in regrowth of hair.

### Example 14

4 golden retriever puppies of 40 days growth were taken for study. They were divided into two groups. First group was given puppy food with omega 3 to 6 ratio 1:10 and the second group was given puppy food with omega 3 to 6 ratio 1:1 by using optimized nutrient oil prepared by a process given in example 2. Soy bean oil with omega 3 to 6 ratio 1:7 was used as fur care oil for the first group and optimized nutrient oil with omega 3 to 6 ratio 1:1 prepared by a process as given in example-3 as fur care oil for the second group. Both the groups were given vaccination and deworming as per standard schedule.

Puppies were evaluated at the end of 30 days and 60 days by weight, fur status and learning ability. The puppies in second group on optimized nutrient oil was showing significantly increased weight gain, fur growth and learning ability compared to the first group.

### Example 15

Three patients with menopausal symptoms were taken for study. The patients were divided into three groups. First patient was advised to take orally estriol one mg as tablet daily. Second patient was advised to apply over skin of thighs and abdomen 0.5 ml twice daily medicated optimized nutrient oil prepared by a process as given in example-7 which contains 0.25 mg estriol/ml, so that 0.25 mg estriol is absorbed daily by the body through skin. Third patient was given oral multivitamin and minerals as tablet.

All the three patients were evaluated after 15 days and 30 days on hot flushes, mood changes and sleep pattern. Patients in first and second group were showing improvement in all the three symptoms but the patient in third group had no improvement and the frequency of hot flushes had increased. Patient in second group had complete relief from hot flushes by 30 days and had better response than the first group. When optimized nutrient oil was used to deliver the hormone estriol to the patient trans dermally it was possible to achieve better symptom control in quarter the dose taken orally. This is due to bypassing the first pass metabolism in the liver when taken trans dermally. High degree of unsaturation of fatty acids in the media promotes easy absorption of the hormone through skin.

### Example 16

One patient with psoriasis who was not able to tolerate oral methotrexate was taken for study. Patient was evaluated with physical examination and laboratory tests including hematology and biochemistry studies.

Patient was asked to apply over the affected skin with optimized nutrient oil prepared by a process as given in example-8, three times a day and medicated optimized nutrient oil containing methotrexate 0.5 mg/ml prepared by a process as given in example-9, one ml weekly. Patient was evaluated every day initially with blood cell count for one week and then weekly for one month and monthly for another two months. There was significant reduction in skin lesions at the end of first week. There were no complications associated with methotrexate as happened while on oral methotrexate for the same patient. By the end of one month the dose of medicated optimized oil was reduced to 0.5 ml weekly and by the end of two months there were no active lesions and it was possible to stop medicated optimized oil. Patient was asked to continue optimized nutrient Oil without methotrexate as in example 8 once daily for another one month. Local delivery of drug methotrexate for psoriasis directly on the affected skin using optimized nutrient oil not only reduces the dose needed to one tenth of the oral dose, but also avoids systemic complications.

### Example 17

20 women between 30-50 years of age who wish to have lighter and smoother skin were taken for study.

Patients were divided into two groups. Both the groups were evaluated for general health assessment with physical check-up and laboratory tests including BRE, URE, blood sugar, vitamin D, LFT and RFT. Both the groups were given standard medical treatment for any abnormality detected in tests. The second group was asked to apply skin care oil prepared by a process as given in example 5 and the first group was asked to apply the regular mineral oil/body care oil all over the body twice daily. The patients were assessed at the end of 30 days and 60 days for color, texture and smoothness.

At the end of 30 days the first group who were on the regular mineral oil/body care oil had no change in skin color but there was a minimal improvement in skin texture and smoothness. But at the end of 30 days for the second group who are on optimized nutrient oil there was significant lightening of the skin along with significant reduction in cellulite on the thighs and buttocks, reduction in wrinkles on face and hand, and improved smoothness and softness of the skin. At the end of 60 days there was no further improvement for the first group who were on mineral oil, but those who were on optimized nutrient oil had significantly fairer, smoother, softer and younger skin.

The optimized nutrient oil with polyunsaturated double bonds is easily absorbed in to the dermal cell and it acts as a media for absorption of vitamins and caffeine in to the cells. The 1:1 ratio of omega 3 & 6 fatty acids balances the inflammatory actions of the pollutants and ultra violet rays on the skin and reduces the melanin production. This optimized nutrient oil has a drying effect and forms a coating over the skin which acts as a protective layer for the skin.

### Example 18

10 postpartum patients 45 days after delivery were taken for study. Patients were divided into two groups. First group were asked to apply coconut oil over the abdomen twice daily. Second group was asked to apply optimized nutrient skin care oil as prepared by a process as given in the example 5. Both the groups were asked to do abdominal wall muscle strengthening exercises. Patients were evaluated after 30 days and 60 days for stretch marks, color of the skin and texture of skin of the abdomen.

At the end of 30 days the second group who were on optimized nutrient oil had marked improvement on stretch marks, color of skin, and texture of skin compared to first group. Again the evaluation at the end of 60 days there was significant improvement for all the three parameters stretch marks, color of skin, and texture of skin of the abdomen for the second group compared to first group.

### Example 19

10 volunteers were taken for a study to evaluate the sunscreen effect of optimized nutrient oil. All were asked to get sun exposure 2 hours every day. Before sun exposure volunteers were asked to apply optimized nutrient skin care oil as prepared by a process as given in the example 5 on the right side of the face and right hand and sunscreen lotion of SPF 50 on the left side of the face and left hand.

Study group were evaluated after 15 days and 30 days. The right side of the face and right hand where the optimized nutrient oil was applied was showing fairer, brighter and smoother skin than the left side where the sun screen lotion of SPF 50 was used.

The better sun protection effect of optimized nutrient oil than a sunscreen lotion of SPF 50 is due to the protective coating of the oil at the skin due to its drying properties and easily absorbable nature of the oil due to the unsaturated fatty acids which protect the skin from the inflammatory effect of ultraviolet rays. DHA in the optimized nutrient oil has strong anti-inflammatory properties which helps fast recovery from sun damage.

### Example 20

4 patients with chronic eczema were selected for study. After regular physical checkup and investigations the patients were divided into two groups. Both groups were given clobetasol propionate+miconazole nitrate cream for application over the affected area twice daily. In addition the second group was asked to take 25 ml per day optimized nutrient oil prepared by a process as given in example-1 as cooking oil and apply optimized nutrient skin care oil as prepared by a process as given in the example 4 over the affected skin after applying the ointment twice daily. The second group was also advised to avoid nuts and oil seeds and cooking oil containing omega 6 fatty acids to keep the omega 3 to 6 ratio around 1:1 in the food.

On evaluation at the end of 10 days both the groups showed significant improvement. In the first group there was significantly more hyper pigmentation on the healing affected area than the second group. Both the groups were asked to stop the pharmaceutical cream, but the second group was asked to continue the optimized nutrient oil as food and skin care oil. At the end of 30 days the first group who were not on optimized nutrient oil had recurrence of eczema but the second group was relieved of eczema and the skin at the affected area showed complete recovery.

### Example 21

10 patients with metabolic syndrome (high blood pressure, abdominal obesity, elevated fasting glucose level, elevated fasting triglyceride level, and reduced HDL) were selected for study. After physical check-up including weight, abdominal girth and blood pressure and investigations including blood sugar, lipid profile, CRP, SGPT and ultrasound examination for fatty liver, the patients were divided into two groups. Both group were asked to continue currently using medication and advised low calorie, low fat, low carbohydrate diet and moderate exercise. In addition the second group was asked to take 25 ml per day optimized nutrient oil prepared by a process as given in example-1 as cooking oil. The second group was also advised to avoid nuts and oil seeds and cooking oil other than 25 ml optimized nutrient oil.

Evaluation at the end of 30 days showed significant reduction in weight, abdominal girth, blood pressure, blood sugar, CRP, SGPT and triglyceride; and an increase in HDL in the second group who were on optimized nutrient oil compared to the first group. 25 ml of optimized nutrient oil as prepared in example 1 will provide around 8.5 g omega 3 fatty acid with an omega 3 to 6 ratio 1:1. This 25 ml oil will give enough essential fatty acids for a day and this high content of omega 3 & 6 fatty acids in 1.25-1:1 ratio is valuable in a calorie restricted diet of metabolic syndrome patients which comprises around 35% of adult population of the modern world. Balance of omega 3 to 6 ratio helps in balancing the ecosanoids mediated inflammatory and immune activities and there by reduces the risk for diseases including heart disease, stroke, cancer, arthritis, asthma, allergy and autoimmune diseases

### Example 22

10 patients with cracked foot were taken for study. They were evaluated with regular health check-up and standard modern medical advice on foot care and cream for cracked foot containing salicylic acid+clobestasol+miconazole twice daily on both feet were given. The patients were asked to apply optimized nutrient oil as prepared by a process as given in the example 5 three times daily on the right foot and coconut oil on the left foot. At the end of 15 days there was significant improvement in both legs but the right leg with optimized oil use was free of cracks and attained smooth skin, but the left was showing dryness and only partial cure. Patients were asked to stop pharmaceutical ointment and continue with optimized nutrient oil application on both feet. At the end of 30 days both legs were free of cracks and the patients were relieved.

The triglycerides with polyunsaturated fatty acid absorbed into skin of the foot is used for the synthesis of cell membrane bilayer which is flexible and allows easy exchange of nutrients and waste products across the cell. The vitamin in the enriched optimized nutrient oil is directly absorbed and available for cellular regeneration. In addition the drying effect of the optimized nutrient oil makes a thin coating over the skin which prevents moisture loss and acts as a moisturizer for the skin. Balanced ratio of omega 3 and omega 6 fatty acids balances the inflammatory and anti-inflammatory activities in the cells and helps healing. DHA in the optimized nutrient oil helps the regeneration of sensory receptors of the skin. The caffeine in this enriched optimized nutrient oil has an action of improving the microcirculation which also helps in healing and improved absorption of nutrients. The special feature of the optimized nutrient oil makes it possible to act as a media for absorption of fat insoluble agents like caffeine also through the skin which opens a wide opportunity of delivering nutrients and medicines to the body through skin which helps in reducing the dose of pharmaceutical agents with the benefit of bypassing the first pass metabolism in liver and associated complication as in example 7 where the delivery of methotrexate for psoriasis and in example 6 in delivery of hormone estrogen for hormone replacement therapy in postmenopausal women.

### Example 23

8 patients who were on treatment for bronchial asthma stabilized on inhalation medication twice daily and monteleukast 5 mg tablets daily, were taken for study. The patients were divided into two groups after standard preliminary physical examination and laboratory investigation. Both the groups were asked to continue the inhaler. The second group was asked to take 25 ml per day optimized nutrient oil prepared by a process as given in example-1 as cooking oil. The second group was also advised to avoid nuts and oil seeds and cooking oil containing omega 6 fatty acids to keep the omega 3 to 6 ratio around 1:1 in the food.

At the end of 15 days both the groups were asked to reduce the inhalation to once daily. At the end of 30 days the second group who were on optimized nutrient oil was comfortable with reduced dose of inhaler, but the first group which was not given optimized nutrient oil was not able to reduce the inhaler due to breathlessness. Both the groups were asked to stop monteleukast but asked to continue inhaler twice daily by the first group and once daily by the second group. At the end of 45 days first group was reporting breathlessness especially on exertion. But the second group who were on optimized nutrient oil was comfortable with half the dose of inhalant, without monteleukast tablets.

The reduction in drugs for the treatment of asthma in the second group who were on optimized nutrient oil was due to high omega 3 fatty acid content with omega 6 and 3 ratio 1:1. This balances the production of omega 6 and 3 mediated ecosanoids, the signaling molecules produced from 20 carbon omega 3 EPA and omega 6 arachidonic acid. Monteleukast is a leucotrins receptor antagonist which, blocks the leucotrins mediated inflammatory process responsible for asthma. When the omega 6 and 3 fatty acids in the diet are balanced, the production of arachidonic acid the precursor for leucotrins is reduced and a balance between inflammatory and anti-inflammatory activity in the body is achieved. This is the reason for better control of bronchial asthma and other inflammatory and immune mediated disorders by the use of optimized nutrient oil.

We have brought out the novel features of the invention by explaining some of the preferred embodiments under the invention, enabling those in the art to understand and visualize our invention. Numerous changes can be made without departing from the concept discussed herein. Hence it is to be understood that within the scope of the claims provided herein, the invention may be practiced otherwise then as specifically described herein.

We claim:

1. An optimized nutrient fatty acid composition capable of using as food, skin care, hair care and media for delivery of drugs and nutrients through mucous membrane, skin and parenterally for humans, pets and farm animals, comprising: a ratio of omega 6 fatty acid to omega 3 fatty acids in the composition ranging from 1:1 to 1:1.25.

2. The optimized nutrient fatty acid composition as claimed in claim 1, comprising: vitamin E in the range of 0.8-4 mg/ml of the composition to protect the unsaturated fatty acids from lipid peroxidation in vitro and in vivo and to avoid rancidity; and the amount of vitamin E needed for the composition is calculated based on the formula (% R1×1+% R2×2+% R3×3+% R4×4+% R5×5+% R6×6) divided by the factor F, wherein % R1 is the percentage of fatty acid with one double bond in the fatty acid chain, % R2 is the percentage of fatty acid with two double bonds in the fatty acid chain, % R3 is the percentage of fatty acid with three double bonds in the fatty acid chain, % R4 is the percentage of fatty acid with four double bonds in the fatty acid chain, % R5 is the percentage of fatty acid with five double bonds in the fatty acid chain, % R6 is the percentage of fatty acid with six double bonds in the fatty acid chain, the factor F is an arbitrary number 150.

3. The optimized nutrient fatty acid composition as claimed in claim 1, comprising: 50-70% of the total fatty acids as essential or semi essential fatty acids; wherein 25-35% as omega 3 fatty acids; 25-35% as omega 6 fatty acids; and stabilized with vitamin E in the range of 0.8-4 mg/ml.

4. The optimized nutrient fatty acid composition as claimed in claim 1, comprising: a) 25-35% of the total fatty acids as omega 3 fatty acids selected from a group consisting of ALA (alpha linoleic acid), SDA (steriodonic acid), ETA (ecosatetraenoic acid), EPA (ecosapentanoic acid), DPA (decosapentaenoic acid), and DHA Decosahexanoic acid); b) 25-35% as omega 6 fatty acids selected from a group consisting of LA (linoleic acid), GLA (gama linoleic acid), DGLA (dihomogama linoleic acid) and AA (arachidonic acid); c) 20-30% mono unsaturated fatty acid (MUFA) selected from a group consisting of oleic acid, palmitoic acid, vaccenic acid and erucic acid; and d) vitamin E in the range of 0.8-4 mg/ml.

5. The optimized nutrient fatty acid composition as claimed in claim 1, comprising: a) 25-35% of the total fatty acids as omega 3 fatty acids selected from a group including ALA (alpha linoleic acid), SDA (steriodonic acid), ETA (ecosatetraenoic acid), EPA (ecosapentanoic acid), DPA (decosapentaenoic acid), and DHA (decosahexanoic acid); b) 25-35% as omega 6 fatty acids selected from a group including LA (linoleic acid), GLA (gamalinoleic acid), DGLA (dihomogamalinoleic acid) and AA (arachidonic acid); c) 15-25% as monounsaturated fatty acids (MUFA) selected from a group including oleic acid, palmitoic acid, vaccenic acid and erucic acid; d) 15-25% as saturated fatty acids; and e) vitamin E in the range of 0.8-4 mg/ml.

6. The optimized nutrient fatty acid composition as claimed in claim 1, comprising: a) the ratio of omega 3 fatty acid to omega 6 fatty acid 1:1; b) 60% of the total fatty acids as essential or semi essential fatty acids, 20% as monounsaturated fatty acids (MUFA)) and 20% as saturated fatty acids and non-fatty acid components; c) 30% omega 3 fatty acids selected from a group consisting of ALA (alpha linoleic acid), SDA (steriodonic acid), ETA (ecosatetraenoic acid), EPA (ecosapentanoic acid), DPA (decosapentaenoic acid), and DHA Decosahexanoic acid); d) 30% omega 6 fatty acids selected from a group consisting of LA (linoleic acid), GLA (gamalinoleic acid), DGLA (dihomogamalinoleic acid) and AA (arachidonic acid); e) 20% as monounsaturated fatty acids selected from a group consisting of oleic acid, palmitoic acid, vaccenic acid and erucic acid; f) 20% as saturated fatty acids and non-fatty acid components; and g) vitamin E in the range of 0.8-4 mg/ml.

7. The optimized nutrient fatty acid composition as claimed in claim 1, wherein the source of fatty acids are selected from a group comprising:
a) a plant source;
b) a genetically modified plant source;
c) an animal source;
d) a genetically modified animal source;
e) a marine life source;
f) a genetically modified marine life source;
g) an algal source;
h) a genetically modified algal source;
i) a fungal source;
j) a genetically modified fungal source;
k) a microorganism; and
l) a genetically modified microorganism.

8. The optimized nutrient fatty acid composition as claimed in claim 1, wherein: a) selection of type fatty acids from a group consisting of ALA (alpha linoleic acid), SDA (steriodonic acid), ETA (ecosatetraenoic acid), EPA (ecosapentanoic acid), DPA (decosapentaenoic acid), DHA (Decosahexanoic acid), LA (linoleic acid), GLA (gamalinoleic acid), DGLA (dihomogamalinoleic acid) and AA (arachidonic acid), oleic acid, palmitoic acid, vaccenic acid, erucic acid and saturated fatty acids; b) selection of the source of fatty acids depends on the oil content and the fatty acid profile of the source; c) selection of different combinations of source materials depends according to the seasonal and geographical availability of fatty acid source.

9. The optimized nutrient fatty acid composition as claimed in claim 1, wherein ALA as omega 3 and LA as omega 6 is selected from one of the following group a) to e) comprising: a) Sunflower Seed, Flax seed and Gingili Seed; b) Soya Beans, Perilla Seed and Olive; c) Perilla Seed, Safflower Seed and Avocado; d) Flax seed, Walnut and Garden Cress Seed; and e) Corn Seed; Sacha Inchi and Macadamia Seed.

10. The optimized nutrient fatty acid composition as claimed in claim 1 wherein ALA+EPA+DHA as omega 3 and LA as omega 6 is selected from one of the following group a) to e) comprising: a) Sunflower Seed, Flax seed, Chia Seed, and Algae oil; b) Soya Beans, Perilla Seed, Garden Cress and Salmon oil; c) Perilla Seed, Safflower Seed, Avocado, and Krill oil; d) Flax seed, Walnut, Gingili Seed, and Sardine oil; and e) Corn Seed, Sacha Inchi, Sea Buckthorn and squid oil.

11. The optimized nutrient fatty acid composition as claimed in claim 1, wherein ALA as omega 3 and LA+GLA as omega 6 is selected from one of the following group a) to e) comprising: a) Sunflower Seed, Flax seed, Gingili Seed and Evening Primrose; b) Soya Beans, Perilla Seed; Olive; Black current; c) Perilla Seed, Water melon Seed, Avocado, High GLA Safflower; d) Flax seed, Walnut, Garden cress, Borage Seed; and e) Corn Seed, Sacha Inchi, Macadamia, Evening Prim rose.

12. The optimized nutrient fatty acid composition as claimed in claim 1, wherein ALA+EPA+DHA as omega 3 and LA+GLA as omega 6 is selected from one of the following group a) to e) comprising: a) Sunflower Seed, Flax seed, Evening Primrose, and Algae oil; b) Soya Beans, Perilla Seed, Black current seed, and Salmon Oil; c) Perilla Seed, Safflower Seed, Hemp seed, and Krill oil; d) Flax seed, Cotton Seed, Borage Seed, and Sardine oil; and e) Corn Seed, Sacha Inchi, Evening primrose, and Squid Oil.

13. The optimized nutrient fatty acid composition as claimed in claim 1, wherein ALA+EPA+DHA as omega 3 and LA+AA as omega 6 is selected from one of the following group a) to e) comprising: a) Sunflower Seed, Flax seed, Gingili Seed, Algae oil, Fungus Mortierella alpine; b) Soya Beans, Perilla Seed, Chia Seed, Salmon Oil, Fungus Mortierella alpina; c) Perilla Seed, Safflower Seed, Avocado, Krill oil, Fungus Mortierella alpine; d) Flax seed, Walnut, Garden cress, Sardine oil, Fungus Mortierella alpine; and e) Corn Seed, Sacha Inch′, Macadamia, Fungi oil (recombinant), Fungus Mortierella alpine

14. The optimized nutrient fatty acid composition as claimed in claim 1, wherein ALA+EPA+DPA+DHA as omega 3 and LA+GLA+AA as omega 6 is selected from one of the following group a) to e) comprising: a) Sunflower Seed, Flax seed, Gingili Seed, Evening Primrose, Algae oil, Fungus Mortierella alpine; b) Soya Beans, Perilla Seed, Chia Seed, Black current seed, Salmon Oil, Micro algae; c) Perilla Seed, Cotton Seed, Avocado, High GLA Safflower, Krill oil, Fungus Mortierella alpine; d) Flax seed, Walnut, Garden cress, Borage Seed, Micro algae, Fungus Mortierella alpine; and e) Corn Seed, Sacha Inchi, Macadamia, Evening primrose, Fungi oil (Recombinant), Fungus Mortierella alpine.

15. A method of preparation of the optimized nutrient fatty acid composition, comprising:
a) selecting the source of fatty acids from a group including a plant source, a genetically modified plant source, an animal source, a genetically modified animal source, a marine life source, a genetically modified marine life source, an algal source, a genetically modified algal source, a fungal source, a genetically modified fungal source, a microorganism, a genetically modified microorganism;
b) cleaning and drying the selected ingredients;
d) dehydrating the ingredients at 40-80 degree centigrade to avoid hydrolytic degradation of unsaturated fatty acids;
e) extracting the oil after cleaning, drying and dehydrating the selected raw materials using known technologies for the selected source materials;
f) analyzing the oil samples of step e) for fatty acid composition;
g) measuring and combining different oils considering the fatty acid analysis data to get the fatty acid composition with omega 6 fatty acid to omega 3 fatty acid ratio ranging from 1:1 to 1:1.25;
h) stabilizing the unsaturated fatty acids by adding vitamin E in the range of 0.8-4 mg/ml of the composition to protect the unsaturated fatty acids from lipid peroxidation in vitro and in vivo and to avoid rancidity;
i) calculating the amount of vitamin E needed using the formula (% R1×1+% R2×2+% R3×3+% R4×4+% R5×5+% R6×6) divided by factor F, wherein % R1 is the percentage of fatty acid with one double bond in the fatty acid chain, % R2 is the percentage of fatty acid with two double bonds in the fatty acid chain, % R3 is the percentage of fatty acid with three double bonds in the fatty acid chain, % R4 is the percentage of fatty acid with four double bonds in the fatty acid chain, % R5 is the percentage of fatty acid with five double bonds in the fatty acid chain, % R6 is the percentage of fatty acid with six double bonds in the fatty acid chain, the factor F is an arbitrary number 150;
j) mixing the fatty acid composition in a stirrer at 200-500 RPM for 2-5 minutes to get an even mixture; and
k) packing the product of step j) in to air tight and opaque containers to avoid contact with oxygen and exposure to light.

16. The optimized nutrient fatty acid composition as claimed in claim 15, wherein:
a) the resultant optimized fatty acid composition as ingredient for the production of lotion, cream and ointment for cosmetic and pharmaceutical use;
b) the resultant optimized fatty acid composition is added with fragrance selected from a group containing different natural essential oil and, or artificial fragrances and used as skin care and hair care preparations;
b) the resultant fragrant optimized nutrient fatty acid composition is further enriched with nutrients and used as skin care and hair care preparations;
c) the resultant fragrant optimized nutrient fatty acid composition is medicated with drugs and, or nutrients for pharmaceutical and cosmetic use.

17. The optimized nutrient fatty acid composition as claimed in claim 15, wherein:
a) optimized nutrient fatty acid composition is subjected to filtration and ultra-purification;
b) mixed with water, lecithin and glycerol to make intra venous lipid emulsion;
c) resultant intravenous lipid emulsion with optimized fatty acid composition, to provide nutritional support for those who cannot take food orally, for removing lipid soluble toxins in the body, as intra venous lipid to prevent reperfusion injury, as intra venous lipid for patient with acute brain injuries, premature infant feeding, immune modulation in recurrent abortions, and as media for delivering drugs and other nutrients parenterally.

18. The optimized nutrient fatty acid composition as claimed in claim 15, wherein: a) fully hydrogenated fat after liquefaction by heating is mixed with the optimized nutrient fatty acid composition to get fat solids and semi solids of the consistency of butter and spread respectively; b) the resultant optimized nutrient fat for use as food, food ingredient and cooking media; c) the resultant optimized fat is mixed with water, emulsifiers and food ingredients to make food products including mayonnaise, margarine, spread and bakery shortening.

19. An optimized nutrient fatty acid composition as claimed above, wherein: it is capable of use as
a) a food;
b) a food ingredient;
c) a food supplement;
d) a cooking oil;
e) a skin care oil;
f) a hair care oil;
g) a carrier oil for nutrients and drugs;
h) a base oil for ointments, creams and lotions for cosmetic and pharmaceutical preparations; and
i) a base oil for preparation of intravenous lipid emulsions.

20. An optimized nutrient fatty acid composition as claimed above, comprising: a means for a) optimization of bodily functions; b) maintenance of health; c) prevention of diseases; d) promotion of healing; e) delay aging; f) treatment of diseases due to imbalance in omega 3 and 6 fatty acid ratio in diet selected from a group consisting of heart disease, dyslipidemia, hypertension, type 2 diabetes, cancer, mental disorders, immunity related diseases, inflammatory diseases, asthma, allergy, eczema, arthritis and psoriasis; and g) nutritional support for pregnant and lactating women, growing children, senior citizens, sports persons, obese and over weight on calorie restricted diet.
